(19) United States
(12) Patent Application Publication
(10) Pub. No.: US 2017/0051354 A1
(43) Pub. Date: Feb. 23, 2017

(54) EPIGENETIC MODIFICATION OF MAMMALIAN GENOMES USING TARGETED ENDONUCLEASES

(71) Applicant: SIGMA-ALDRICH CO. LLC, St. Louis, MO (US)

(72) Inventors: Gregory D. DAVIS, St. Louis, MO (US); Qiaohua KANG, St. Louis, MO (US)

(73) Assignee: SIGMA-ALDRICH CO. LLC, St. Louis, MO (US)

(21) Appl. No.: 15/306,720

(22) PCT Filed: Apr. 24, 2015
(86) PCT No.: PCT/US15/27541

§ 371 Date: Oct. 25, 2016

Related U.S. Application Data
(60) Provisional application No. 61/985,205, filed on Apr. 28, 2014.

(51) Int. Cl. C12Q 1/68; C12N 15/90

(52) U.S. Cl. CPC C12Q 1/6883; C12N 15/907; C12Q 2600/118; C12Q 2600/154; C12Q 2600/166; C12N 2510/00

(57) ABSTRACT

The present disclosure provides genetically engineered cell lines comprising chromosomally integrated synthetic sequences having predetermined epigenetic modifications, wherein a predetermined epigenetic modification is correlated with a known diagnosis, prognosis or level of sensitivity to a disease treatment. Also provided are kits comprising said epigenetically modified synthetic nucleic acids or cells comprising said epigenetically modified synthetic nucleic acids that can be used as reference standards for predicting responsiveness to therapeutic treatments, diagnosing diseases, or predicting disease prognosis.

### FIELD OF THE INVENTION

The present disclosure relates to epigenetic modification of genomic sequences. In particular, the present disclosure relates to genetically engineered cell lines comprising chromosomally integrated nucleic acid sequences having predetermined epigenetic modifications.

### BACKGROUND

Aberrant gene function and altered patterns of gene expression are key features of numerous diseases and conditions. Growing evidence indicates that epigenetic alterations participate with genetic aberrations to cause dysregulation. Recent advances in the detection and quantification of epigenetic modifications in genomic DNA are leading to a new generation of diagnostic and prognostic assays for numerous diseases, for example, cancer. In addition, epigenetic alterations have been shown to correlate with the level of sensitivity to certain disease treatment regimens and as a result are being used in treatment decisions.

Despite advances in the development of diagnostic and prognostic assays and treatment protocols based on epigenetic modifications, there is currently a lack of cellular reference standards available for assessing epigenetic alteration status.

### SUMMARY OF THE INVENTION

In recent years, there has been interest in using advances in genome editing technology to modify the genome of human cells to create disease models mirroring genotypes observed in clinical samples. In addition to analyzing phenotypes for research purposes, genetically or epigenetically engineered cells can also be used as genotyping references or standards for clinical assays. Among the advantages of such engineered reference cell lines are that: (1) they provide a DNA assay template within a native cellular and genomic context that undergoes all subsequent diagnostic processing steps of cell lysis (or formalin-fixed, paraffin-embedded (FFPE) extraction), DNA isolation, and amplification, and (2) the genetic or epigenetic alteration can be modeled into a cell type that is stable and provides large quantities of the genomic DNA.

One aspect of the present disclosure provides a genetically engineered cell line comprising at least one chromosomally integrated nucleic acid having a predetermined epigenetic modification, wherein the predetermined epigenetic modification is correlated with a known diagnosis, prognosis, and/or level of sensitivity to a disease treatment. In some aspects, the epigenetic modification is a modification of a cytosine, for example methylation of a cytosine. In certain aspects, the epigenetically modified nucleic acid has substantial sequence identity to that of a control element or a portion of a control element of a gene associated with a disease. In other aspects, the epigenetically modified nucleic acid has substantial sequence identity to that of a coding region or a portion of a coding region of a gene associated with a disease. Examples of genes having epigenetic alterations associated with disease and/or disease treatment outcome are provided herein. In some aspects, the epigenetically modified nucleic acid can replace the endogenous chromosomal sequence from which the epigenetically modified nucleic acid is derived. Thus, the native epigenetic status of the endogenous chromosomal sequence can be changed to the predetermined epigenetic status of the inserted synthetic nucleic acid. Alternatively, the nucleic acid having the predetermined epigenetic modification can be inserted at a locus, such as AAVS1, CCR5, or SOSA26, possessing adjacent insulating elements or other elements that assist in maintaining the predetermined epigenetic modification status of the inserted nucleic acid. In such instances, the endogenous chromosomal sequence corresponding to the synthetic epigenetically modified sequence can be inactivated or deleted. The epigenetic modification status of the integrated nucleic acid can be stable or metastable. The nucleic acid having epigenetic modification can be inserted into the chromosomal location of interest using a targeting endonuclease. The targeting endonuclease can be a zinc finger nuclease, a CRISPR-based endonuclease, a meganuclease, a transcription activator-like effector nuclease (TALEN), an I-TevI nuclease or related monomeric hybrid, or an artificial targeted DNA double strand break inducing agent. Optionally, cells comprising integrated epigenetically modified sequences can further comprise at least one a nucleic acid encoding a recombinant protein. The engineered cell line can be a mammalian cell line, including a human cell line.

The engineered cells or cell lines comprising integrated nucleic acids having predetermined epigenetic modification have several uses. In certain aspects, engineered cells harboring insertions of synthetic sequences that alter the epigenetic status of regulatory regions can be used to control or alter gene expression. For example, cells having insertion of epigenetically modified synthetic sequence (such as methylated sequence) in addition to or in place of endogenous regulatory chromosomal sequence not normally modified (i.e., not normally methylated or hypermethylated) can be used to alter gene expression. Conversely, the replacement of endogenous regulatory sequence known to have epigenetic modification with a synthetic sequence devoid of epigenetic modification or the insertion of synthetic sequence devoid of epigenetic modification can be used to alter gene expression. In another aspect, engineered cells having insertion of epigenetically modified sequence can be used to analyze the epigenetic stability of a modified sequence in a cell based on a priori knowledge of the epigenetic modification pattern or status of the inserted sequence. In other aspects, engineered cells having insertion of epigenetically modified sequence can be used as reference cell lines in diagnostic and/or prognostic assays by virtue of their known or predetermined epigenetic modification status, which allows them to serve as diagnostic and/or prognostic standards in such assays. In further aspects, cells having insertion of epigenetically modified sequence can be used in assays to assess the suitability of drug treatment regimens (see FIG. 3). Thus, the epigenetically modified sequences and cells containing said sequences can be used as reference standards in assays for diagnosing disease (such as cancer), predicting the outcome of disease, monitoring disease behavior, and measuring response to targeted therapy.

The present disclosure also provides kits for predicting responsiveness of a disease in a subject to a therapeutic treatment, diagnosing a disease in a subject, or predicting the prognosis of a disease in a subject, wherein a kit comprises at least one nucleic acid having predetermined epigenetic modification that is correlated with a known diagnosis, prognosis, or level of sensitivity to a disease treatment.

Additional aspects and iterations of the disclosure are described in more detail below.

### BRIEF DESCRIPTION OF THE FIGURES

FIG. 1A diagrams the targeted integration of synthetically methylated DNA using zinc finger nuclease (ZFN) technology. Diagrammed is cleavage of the AAVS1 target site by a targeted ZFN and integration of the donor sequence comprising a 19 bp MGMT gene fragment into the target site by a cellular DNA repair process.

FIG. 1B diagrams the three different predetermined methylation patterns. The * symbols refer to the four CpG sites (i.e., 1, 2, 3, 4) within the MGMT gene fragment.

FIG. 2 illustrates the stability of the synthetic methylation patterns over time. Plotted is the methylation percentage at each CpG site in the MGMT gene fragment in colony #1 or colony #7 after 49 days or 80 days in culture.

FIG. 3 presents a schematic diagram showing use of MGMT promoter methylation status for determining whether to prescribe temozolomide for treatment of glioblastoma.

### DETAILED DESCRIPTION OF THE INVENTION

The present disclosure provides synthetic nucleic acids comprising epigenetic modifications, as well as engineered cells or cell lines comprising said synthetic sequences as detailed herein. Epigenetic modifications are increasingly appreciated for their effects on disease phenotype, particularly with regard to cancer. Cells comprising synthetic sequences having epigenetic modifications according to the present disclosure may be modeled into a cell type that is stable and provides large quantities of genomic DNA available for research and clinical purposes. The cells of the present disclosure can also serve as physiologically relevant and robust cellular reference standards for assays involving epigenetic modification in mammalian cells. Such standards are useful in diagnostic and prognostic assays, as well as in the assessment of treatment regimens in individual subjects.

### I. Nucleic Acids Having Predetermined Epigenetic Modifications

(a) Epigenetic Modifications

The present disclosure provides nucleic acids having predetermined epigenetic modifications, wherein the predetermined epigenetic modification is correlated with a known diagnosis, prognosis or level of sensitivity to a disease treatment. In general, the epigenetically modified nucleic acids are synthetic nucleic acids in which the epigenetic modification is chemically produced. In one aspect, the epigenetic modification is a cytosine modification. The cytosine modification can be any such modification known to one of ordinary skill in the art, such as methylation of cytosine including 5-methylcytosine (5mC), 3-methylcytosine (3mC), and 5-hydroxymethylcytosine), 5-formylcytosine (5fC), and 5-carboxylcytosine (5caC). In one embodiment, the epigenetic modification is methylation of a cytosine, including for example 5-methylcytosine (5mC), 3-methylcytosine (3mC), and 5-hydroxymethylcytosine. In one instance, the modified cytosine is 5-methylcytosine. In one embodiment, the methylated cytosine is present in a CpG, which may be present in individual CpG sites or grouped in a cluster of CpGs, referred to as a CpG island.

In one aspect, the cytosine modification is a modification of the methylation status cytosine, which includes both methylation and hydroxymethylation. Methylation status refers to features such as the number or percentage of methylated cytosine residues in a sequence, i.e., methylation level, or the pattern of methylated residues within a sequence. The predetermined methylation status may be tailored based on the gene of interest as well as the intended use of the output. In some aspects, a cellular reference standard desirably exhibits high levels of methylation, or alternatively, low or absent methylation may be preferred. It will be understood that several different criteria are known to those of ordinary skill in the art for calculating methylation level. For example, methylation level may be the percentage of methylated residues in a particular CpG island, or an average of methylation over several CpG islands. It will be understood by those of skill in the art that features other than CpG islands may also be methylated, such as sequences generally having the form CHG and CHH, where H is A, C, or T (e.g. CAG, CTG, CAA, CAT, etc.). The methylation level may also be measured globally across the entire chromosomal sequence.

A nucleic acid may be described as methylated or non-methylated using any suitable convention. For example, one of ordinary skill in the art may consider a nucleic acid to be methylated if at least 10% of CpG residues are methylated in a particular island, and non-methylated if less than 10% of CpG residues are methylated. Of course, if features other than CpG residues are methylated, such methylations may also be included in the calculation as appropriate. Alternatively, a nucleic acid may be described as having a methylation level of a certain percentage, e.g., about 1%, 5%, 10%, 15%, 20%, 25%, 30%, 35%, 40%, 45%, 50%, 55%, 60%, 65%, 70%, 75%, 80%, 85%, 90%, 95%, or 100% of cytosine residues are methylated. It will be further understood that intervening values are contemplated. Nucleic acids having 0% or approximately 0% methylation are also contemplated. It may further be expedient to one of ordinary skill in the art to identify methylation levels qualitatively, e.g., “high,” “moderate,” or “low.” Such terms may be readily defined as necessary with reference to (1) levels of methylation found in endogenous chromosomal sequence of normal or healthy cells, (2) levels of methylation found in endogenous chromosomal sequence of cells having a particular phenotype, including but not limited to abnormal or disease phenotype and/or phenotype of drug treatment sensitivity or resistance, (3) levels of methylation found in endogenous chromosomal sequence corresponding to normal level of gene expression; and (4) levels of methylation found in endogenous chromosomal sequence corresponding to abnormal level of gene expression (i.e., over- or under-expression).

Methylation status may refer to a particular pattern of methylation in a nucleic acid of interest, alone or in combination with the percentage of methylated residues. It will be understood, however, that one of ordinary skill in the art is capable of interpreting the similarities and differences between methylation of the nucleic acids of the present disclosure and methylation of endogenous chromosomal sequences detected in a sample taken from a subject, as well as previously known or established methylation levels and/or patterns.

Methods for determining the level and/or pattern of methylation are known in the art and include, for example, digital quantification (Li et al., Nature Biotechnology, 27:858-863 (2009) and supplementary materials (doi:10.1038/nbt.1559)); methylation-specific PCR (MSP) which involves reacting the chromosomal sequence with sodium bisulfite followed by PCR (Herman et al., PNAS, 93: 9821-9826 (1996); Gonzalgo et al., Cancer Res., 57: 594-599 (1997); Hegi et al., Clin. Cancer Res., 10:1871-1874 (2004)); whole genome bisulfate sequencing (BS-Seq); HELP assay which involves restriction enzymes' ability to differentially cleave methylated and unmethylated DNA (using methylation-sensitive restriction enzyme or methylation-dependent restriction enzyme); ChIP-on-chip assay which is based on the ability of antibodies to bind to DNA-methylation-associated proteins; restriction landmark genomic scanning which is similar to the HELP assay (Hayashizaki et al., Electrophoresis, 14:251-258 (1993); Costello et al., Nat Genet, 24:132-138 (2000)); methylated DNA immunoprecipitation (MeDIP) which is used to isolate methylated DNA fragments; pyrosequencing of bisulfate treated DNA (Tost et al., BioTechniques, 35:152-156 (2003)); MS-qFRET (Bailey et al., Genome Res, 19:1455-1461 (2009)); quantitative differentially methylated regions (QDMR); methyl sensitive southern blotting (similar to HELP assay); MethylLight, MS HRM, MethylMeter® (McCarthy et al., “MethyMeter®: A Quantitative, Sensitive, and Bisulfate-free Method for Analysis of DNA Methylation” in Biochemistry, Genetics and Molecular Biology, Chapter 5 “DNA Methylation—from Genomics to Technology; eds T. Tatarinova and O. Kerton, In-Tech, March 2012, pp. 93-116); GLIB (Pastor et al., Nature, 473: 394-397 (2011)); anti-CMS (Pastor et al., Nature, 473: 394-397 (2011)); and use of methyl CpG binding proteins which is used to separate native DNA into methylated and unmethylated fractions. Other methods of detecting DNA methylation, including 5-hydroxymethylcytosine, are described in Szwagierczak et al., Nuc. Acids Res., 38:e181 (2010).

(b) Sequence of the Nucleic Acids Having Predetermined Epigenetic Modifications

A nucleic acid with the predetermined epigenetic modification disclosed herein generally has a nucleotide sequence with substantial sequence identity to that of a transcriptional control element, a portion of a transcriptional control element, a coding region, or a portion of a coding region of a gene of interest, wherein the gene of interest is associated with a disease or a disorder. As used herein, the phrase “substantial sequence identity” refers to sequences having at least about 75% sequence identity. In various aspects, the synthetic chromosomal sequences having epigenetic modification can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, or 100% sequence identity with the gene of interest.

In some aspects, the nucleic acid having a predetermined epigenetic modification has substantial sequence identity to that of a transcriptional control element associated with a gene of interest. The control element can be a promoter, an enhancer, a silencer, a locus control element, or any sequence that regulates transcription of a gene. The transcriptional control element can be located upstream, downstream, or within the coding or non-coding (e.g., intron) region of a gene of interest. In specific aspects, the control element is a promoter or part of a promoter located upstream of the transcription start site or within the 5′ region of the gene of interest. In some aspects, epigenetic modification (e.g., cytosine methylation) of the control element completely or partially silences transcription of the gene.

In other aspects, the nucleic acid having a predetermined epigenetic modification has substantial sequence identity to that of a coding region (i.e., one or more exons) or a portion of a coding region of a gene associated with a disease. In one embodiment, the nucleic acid having a predetermined epigenetic modification is hypermethylated compared to the corresponding native or endogenous chromosomal sequence (i.e., the corresponding endogenous sequence in a normal or non-diseased cell or the corresponding endogenous sequence found during normal gene expression (as opposed to over- or under-expression)). In another embodiment, the nucleic acid having a predetermined epigenetic modification is hypomethylated compared to the corresponding native or endogenous chromosomal sequence. Chromosomal regions including exons and introns are known to modulate gene expression via methylation of CpG locations (which may or may not be present as CpG islands). Examples of genes with known exonic and intronic methylation responses include MGMT and CXCR4, among numerous others as provided herein.

In some aspects the nucleic acid having a predetermined epigenetic modification is derived from a gene associated with a disease. Genes of interest include those known to have epigenetically modified sequences and which are associated with diseases such as cancer, autoimmune diseases (such as Type 1 Diabetes, inflammatory bowel disease), inflammatory diseases (such as asthma), metabolic disorders, autism spectrum disorder, and other conditions associated with aberrant gene expression. Particular genes of interest include MGMT, BRCA1, BRCA2, Septin9, PITX2, GSTP1, APC, RASSF1, HER2, P15INK4B, p16INK4A, Rb, E-cad, as well as other genes described in this section. In addition, a non-limiting listing of genes of interest is provided at Table A below.

The genes described herein include genes which are known to be completely or partially silenced by epigenetic modification in the promoter region, such as by aberrant DNA methylation (Jones et al., Cell, 128: 683-692 (2007); Jones et al., Nat. Genet., 21:163-167 (1999); Jones et al., Nat. Rev. Genet., 3, 415-428 (2002)). For example, hypermethylation, in particular high levels of 5-methylcytosine, is one of the major epigenetic modifications that repress transcription via the promoter region, thereby preventing expression of the affected genes. It is well-known that certain cancers are associated with hypermethylated promoter regions of genes, such as tumor suppressor genes (e.g., Rb, p16ink4a, p15ink4b, p73, APC, and VHL), transcription factor genes (e.g., GATA-4, GATA-5, HIC1, and E-cadherin), DNA repair genes (e.g., BRCA1, WRN, FANCF, RAD51C, MGMT, MLH1, MSH2, NEIL1, FANCB, MSH4, ATM, and GSTP1), genes involved in cell-cycle regulation (e.g., p16ink4a, p15ink4b, p14arf, and CDKN2B), genes involved in apoptosis, genes involved in metastasis and invasion (e.g., CDH1, TIMP3, and DAPK), and metabolic enzyme genes. For example, breast, ovarian, gastrointestinal (stomach and colon), pancreatic, liver, kidney, colorectal, lung, bladder, cervical, brain, glioma, leukemia, melanoma, prostate, and head and neck cancers are associated with hypermethylated promoter regions of BRCA1, WRN, FANCF, RAD51C, MGMT, MLH1, MSH2, NEIL1, FANCB, MSH4, Rb, p16ink4a, p15ink4b, p73, APC, VHL, GATA-4, GATA-5, HIC1, E-cadherin, p14arf, CDH1, TIMP3, DAPK, and ATM (i.e., breast—GSTP1, BRCA1, p16ink4a, WRN; ovarian—BRCA1, WRN, FANCF, GSTP1, p16ink4a, RAD51C; colorectal—MGMT, APC, WRN, MLH1, p16ink4a, p14arf, MSH2; head and neck—MGMT, MLH1, NEIL1, FANCB, MSH4, p16ink4a, DAPK, ATM; bladder—p16ink4a, DAPK; cervical—p16ink4a; melanoma—p16ink4a; glioma—p16ink4a; gastrointestinal—p16ink4a, p14arf, MLH1, MGMT, APC; liver—GSTP1; prostate—GSTP1; lung—DAPK, MGMT, p16ink4a). A profile of gene promoter hypermethylation in several different genes across numerous human tumor types is reported in Esteller et al., Cancer Res, 61: 3225-3229 (2001), as well as the many references cited therein. See also, Baylor, Nature Clinical Practice Oncology, 2: S4-S11 (2005).

The genes described herein also include genes in which epigenetic modification in the promoter region, such as aberrant DNA methylation, has been shown to be associated with a particular prognosis or susceptibility to certain treatment regimens, such as certain chemotherapies. For example, methylation of the promoter of mgmt has been correlated with responsiveness to temozolomide. See, e.g., Hegi et al., Clin. Cancer Res. 10(6):1871-4 (2004); Hegi et al., New England J. Med. 352(10): 997-1003 (2005); Boots-Sprenger et al., Modern Pathol. 26(7): 922-9 (2013). Likewise, methylation of brca1 and brca2 promoters has been examined as part of an established diagnostic protocol for determining breast cancer prognosis. See, e.g., Abkevich et al., Br. J. Cancer, 107(10): 1776-82 (2012). Additionally, a methylation assay for Septin9 has been adopted for pathologic evaluation of colorectal cancer. See, e.g., Grutzmann et al., PLos One, 3(11):e3759 (2008). Also, methylation of the E-cadherin promoter is associated with decreased tumor suppression ability and increased likelihood of metastasis. See, e.g., Graff et al., Cancer Res. 55(22): 5195-9 (1995).

Other genes provided herein include genes in which global hypomethylation is associated with the development and progression of cancer. For example, loss of 5-hydroxymethylcytosine is an epigenetic hallmark of melanoma (Lian et al., 2012, Cell, 150:1135-1146); global hypomethylation is linked with formation of repressive chromatin domains and gene silencing in breast cancer (Hon et al., 2012, Genome Res 22(2); 246-58); and global hypomethylation is observed in human colon cancer tissues (Hemandez-Blazquez et al., 2000, Gut 47:689-93).

Genes of interest also include genes associated with the occurrence and/or severity of autism spectrum disorder (ASD). While heritability estimates for ASD are high, clear differences in symptom severity between ASD-concordant monozygotic twin pairs indicates a role for non-genetic epigenetic factors in ASD etiology. (See C. Wong et al., Mol. Psychiatry 2013 (1-9), advanced online publication Apr. 23, 2013; doi: 10.1038/mp.2013.41). Such genes include for example MBD4, AUTS2, MAP2, GABRB3, AFF2, NLGN2, JMJD1C, SNRPN, SNURF, UBE3A, KCNJ10, NFYC, PTPRCAP, RNF185, TINF2, AFF2, GNB2, GRB2, MAP4, PDHX, PIK3C3, SMEK2, THEX1, TCP1, ANKS1A, APXL, BPI, EFTUD2, NUDCD3, SOCS2, NUP43, CCT6A, CEP55, FCJ12505, SRF, DNPEP, TSNAX, FERD3L, RCN2, MBTPS2, PKIA, DAPP1, CCDC41, HOXC5, RPL14, PSMB7, TAF7, INHBB, HNRPA0, MC3R20, BDKRB1, FDFT1, RAD50, 21cg03660451, RECQL5, ZNF499, ARHGAP15, PTPRCAP, C18orf22, RAFTLIN, C14orf143, SUPT5H, ANXA1, C16orf46, GAPDH, FKBP4, LOC112937, SLC30A3, ZNF681, SELENBP1, ELA2, DUSP2, CDC42SE1, PNMA2, POU4F3, DIDO1, SCUBE3, CASP8, THRAP5, ETS1, PXDN, TMEM161A, HOXA9, C11orf1, MGC3207, OR2L13, C19orf33, P2RY11, NRXN1, and ISL1.

Table A lists exemplary genes.

TABLE A
Genes of Interest

| | | | | | |
|---|---|---|---|---|---|
| 21cg03660451 | ABCB | ABCD1 | ABCG2 | ABHD16A | ACSL5 |
| ACTA2 | ADH1B | ADM | ADRB2 | AFF2 | AFF2 |
| AGT | AGTR1 | AK2 | ALCAM | ALDH1A3 | ALDH7A1 |
| ANKS1A | ANXA1 | AOX1 | APC | ApoE | APXL |
| AR | AREG | ARHGAP15 | ATG7 | ATM | ATP1B1 |
| AUTS2 | AX2R | AXIN1 | AXIN2 | BACH1 | BAK1 |
| BAP1 | BAX | BCAS1 | BCL2L14 | BDKRB1 | BDNF |
| BECN1 | BIRC5 | BLT1 | BMP4 | BNC1 | BPI |
| BRCA1 | BRCA2 | BRMS1 | BTG2 | BTK | C10orf99 |
| C14orf143 | C16orf46 | C18orf22 | C1GALT1C | C21orf56 | CA9 |
| CACNA1C | CADM4 | CALCA | CALCR | CASP8 | CCDC41 |
| CCDC88C | CCR5 | CCT6A | CD11a | CD177 | CD31 |
| CD68 | CDC42SE1 | CDH1/ECAD | CDH13 | CDH5 | CDKN1C |
| CDS1 | CDS2 | CDX2 | CEBPa | CELF2 | CEP55 |
| CETP | CHD5 | CHFR | CHGA | CIP29 | CITED2 |
| CLDN4 | CLOCK | C-Myc | C-myc | COASY | COL14A1 |
| COL18A1 | COL1A1 | COL1A2 | COL6A1 | CRBP | CREB3L3 |
| CRYA1 | CSPG2 | DAPK | DAPP1 | DAXX | DBC1 |
| DCC | DDIT4L | DDK2 | DDK4 | DEAF-1 | DGKZ |
| DHCR7 | DIDO1 | DIF2 | Dio3 | DIRAS3 | DKK3 |
| DLC1 | DNAJC24 | DNAPKC | DND1 | DNMT3A | DNMT3B |
| DOA | DPH1 | DUOX2 | DUSP2 | EGAD | ECRG4 |
| EDG1 | EFEMP1 | EFNB3 | EFS | EFTUD2 | EGR |
| EGR4 | EHF | EIF4E | ELA2 | EPHB1 | EPHB2 |
| ERAP1 | ERBB2 | EREG | ERVWE1 | ESO | ESR1 |
| ESR2 | ETS1 | EZH2 | Fact2 | FAM62C | FANCB |
| FANCF | FBXO32 | FCJ12505 | FDFT1 | FKBP4 | FMR1 |
| FOL2 | FOXE1 | FOXI1 | FOXP3 | FRK | FRM1 |
| FTO | FYN | FZD9 | G6PD | GABRB3 | GAD1 |
| GADD45A | GAPDH | GAS | GATA1 | GATA2 | GATA3 |
| GATA4 | GATA6 | GC-1 | GCR | GLI2 | GLS2 |
| GNAS | GNB2 | GNB3 | GNPAT | GPMB | GPNMB |
| GRB2 | GRIK2 | GRS16 | GSTM1 | GSTM2 | GSTP1 |
| GUL1 | H19 | HAND1 | HCG25 | HER2 | HERVE |
| HES4 | HLA-DR51 | HLA-DRA | HLADRB1*1501 | HMGCS2 | HNRPA0 |
| HOXA4 | HOXA9 | HOXB3 | HOXB4 | HOXC5 | HOXD10 |
| HRES1 | HSD17B10 | HSPA9B | HSPB6 | HTR2A | HUMARA |
| IF127 | IG-DMR | IGF1 | IGF2 | IGF2DMR0 | IGF2R |
| IGFAS | IGFBP1 | IGFBP2 | IGFBP3 | IGFBP6 | IGFBP7 |
| IL-10 | IL-13 | IL-15 | IL-16 | IL-17a | IL-18 |
| IL-1B | IL-2 | IL-27RA | IL-4 | IL-6 | IL-7R |
| IL-8 | IL-8RA | INCA1 | INFG | INFGR2 | ING4 |
| INHBB | INSR | IRF8 | ISL1 | ITGB3 | JMJD1C |
| JPH4 | KCNJ10 | KCNMA1 | KCNQ1 | KIR18 | KIR2DL4 |
| KITLG | KLF4 | KLHL3 | KLK2 | Klotho | KRT13 |
| KYNU | Lactoferin | LAG3 | LCN2 | LDHA | LDHC |
| LEP | LET-7A-3 | LGAL | Lin28A | LINE1 | LLGL2 |
| LOC112937 | LOX | LTA | LTA | LTA4H | LTC4S |
| LXN | LY86 | MAGE-3 | MAGEA1 | MAGEL2 | MAGEL3 |
| MaoA | MAP2 | MAP4 | MBD4 | MBTPS2 | MC3R20 |
| Meg3 | MGMT | MLH1 | MN1 | MOBKL2B | MSF |
| MSH2 | MSH4 | MSX1 | MT1G | MTHFR | NANOG |
| NAT1 | NAT2 | NBL2 | NDN | NEFL | NEIL1 |
| NFIB | NFIX | NFYC | NID1 | NLGN2 | NLRP1 |
| NNAT | NOLA1 | NOS2A | NOTCH1 | NOXA1 | NPFFR2 |
| NPM2 | NR1I3 | NRG1 | NUDCD3 | NUP43 | OAS2 |
| OBFC2B | OCT4 | OFD1 | ORAI2 | OTOS | OXTR |
| P14ARF | P14ARF | P15 | P15INK4B | P16 | p16INK4A |
| P21 | P21(CDKN1A) | P73 | PAD | PAI-1 | PAWR |
| | | | | (SERPINE1 | |
| PAX6 | PCDH7 | PDCD5 | PDE11A | PDGFRA | PDHX |
| PDK1 | PDLIM4 | PDX1 | PER1 | PER2 | PGF |
| PGR | PHF11 | PIGR | PIK3C3 | PIK3R1 | PITX1 |
| PITX2 | PKIA | PLCB1 | PLS3 | PNLIPRP3 | PNMA2 |
| PNPLA1 | PNPLA3 | POMC | POU4F3 | PPARGC1A | PPARy |
| PRCH | PRF1 | PRRC2A | PSMB7 | PTEN | PTGER2 |
| PTGS2 | PTH | PTPN6 | PTPRCAP | RAD15C | RAD50 |
| RAFTLIN | RARA | RARB | RARRES1 | RASGRF | RASSF1 |
| Rb | RBL2 | RCN2 | RECQL5 | REELIN | REST |
| RGS5 | RIMBP2 | RNF185 | RPL14 | SCUBE3 | SELENBP1 |
| Septin9 | SFRP1 | SFRP2 | SFRP4 | SFRP5 | SGCE |
| SH3BP2 | SIX3 | SLC17A6 | SLC2A3 | SLC30A3 | SLC39A5 |
| SLC4A3 | SLC5A1 | SLC6A3 | SLC6A4 | SLC9A1 | SMEK2 |
| SNCA | SNRPN | SNTB1 | SNURF | SOCS2 | SOD1 |
| SOD2 | SOX1 | SOX2 | SOX9 | SP140 | SPARC |
| SPI1 | SPRY4 | SRFTSNAX | STAT1 | STAT3 | STAT4 |
| | | FERD3L | | | |
| SULF1 | SUM03 | SUPT5H | TAF7 | TAGLN3 | TCF7 |
| TCF7L2 | TCP1 | TEPP | TERC | TERT | TF |
| TFPI2 | TGFA-308 | TGFBI | THEX1 | THRAP5 | THY |
| TIMP3 | TINF2 | TMCO3 | TMEM18 | TMEM212 | TMS |
| TNFA | TNFRSF10C | TNFRSF10D | TNFRSF25 | TNFSF15 | TNFSF6(FAS) |
| TNFSF7 | TPH2 | TRIM29 | TRIM3 | TRIM31 | TRIM71 |
| TRPA1 | TRPV1 | TRPV3 | TSP50 | TUBB2 | TUSC1 |
| TWIST | UBA7 | UBE3A | UBE4A | UBE4A | UCP2 |
| UGT15MP | UGT17MP | UHRF1 | UMPK | UQCRH | UST |
| VAV1 | VDR | VEGRA | VHL | VLDLR | WDFY1 |
| WRN | ZNF499 | ZNF681 | | | |

(c) Types of Nucleic Acids

The nucleic acids having predetermined epigenetic modification disclosed herein can be RNA, DNA, single-stranded, double-stranded, linear, or circular. In iterations in which the epigenetically modified nucleic acids are double-stranded, the epigenetic modification patterns can be the same or different on the two strands. In some embodiments, both strands can lack the epigenetic modification. In other embodiments, one of the two strands can have the epigenetic modification (i.e., hemi-modified). In further embodiments, both strands can have the epigenetic modification (i.e., duplex-modified). In some instances, the nucleic acids having predetermined epigenetic modification can be a single-stranded, linear molecule, e.g., an oligonucleotide. In other instances, the epigenetically modified nucleic acid can be a double-stranded, linear molecule. Double-stranded, linear nucleic acids can be prepared by the annealing of two complementary single-stranded nucleic acids, or such nucleic acids can be prepared via enzymatic cleavage of longer double-stranded nucleic acids. In some aspects, double-stranded, linear nucleic acids can have overhangs that are compatible with overhangs created by a targeted endonuclease. (As detailed below, targeting endonucleases can be used to insert a nucleic acid having a predetermined epigenetic modification at a specific targeted location in the genome of a cell.) The overhangs can be one, two, three, four, five or more nucleotides in length. In other iterations, some or all of the nucleotides in linear (single- or double-stranded) nucleic acid having epigenetic modification can be linked by phosphorothioate linkages. For example, the terminal two, three, four, or more nucleotides on either end or both ends can have phosphorothioate linkages. In other aspect, the epigenetically modified nucleic acids can be circular. For example, the nuclide acid having predetermined epigenetic modification can be part of a larger polynucleotide, e.g., a plasmid vector, as described in more detail below.

The length of the nucleic acids having epigenetic modification can vary. In general, the epigenetically modified nucleic acid can range in length from about 5 nucleotides (nt) or base pair (bp) to about 200,000 nt/bp. In certain embodiments, the epigenetically modified nucleic acid can range in length from about 5 nt/bp to about 200 nt/bp, from about 200 nt/bp to about 1000 nt/bp, from about 1000 nt/bp to about 5000 nt/bp, from about 5,000 nt/bp to about 20,000 nt/bp, or from about 20,000 nt/bp to about 200,000 nt/bp.

In some aspects, the epigenetically modified nucleic acid can further comprise at least one flanking sequence. The flanking sequence can be upstream, downstream, or both. In one aspect, the epigenetically modified nucleic acid can be flanked by an upstream and/or downstream sequence comprising a restriction endonuclease site. As mentioned above, the epigenetically modified nucleic acid can be flanked (upstream, downstream, or both) by an overhang that is compatible with an overhang created by a targeting endonuclease. In additional aspects, the epigenetically modified nucleic acid can be flanked (upstream, downstream, or both) by at least one insulating element, which can stabilize the epigenetic modification of the epigenetically modified nucleic acid. Insulating elements are known in the art, see, e.g., West et al. Genes & Dev. 16:271-88 (2002); Barkess et al., Epigenomics 4(1):67-80, (2012).

In further aspects, the epigenetically modified nucleic acid can be flanked (upstream, downstream, or both) by a sequence having substantial sequence identity with a sequence on one side of a target site that is recognized by a targeting endonuclease. For example, the epigenetically modified nucleic acid can be flanked by an upstream sequence and a downstream sequence, each of which has substantial sequence identity to a sequence located upstream or downstream, respectively, of a target site that is recognized by a targeting endonuclease. In such cases, the epigenetically modified nucleic acid can be inserted into a targeted chromosomal location by a homology-directed process. As described above, the phrase “substantial sequence identity” refers to sequences having at least about 75% sequence identity. Thus, the upstream and downstream sequences flanking the epigenetically modified nucleic acids can have about 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, or 99% sequence identity with sequence upstream or downstream to the targeted site. In a non-limiting example, the upstream and downstream sequences flanking the epigenetically modified nucleic acids can have about 95% or 100% sequence identity with chromosomal sequences upstream or downstream, respectively, of the targeted site.

In some aspects, the upstream sequence may share substantial sequence identity with a chromosomal sequence located immediately upstream of the targeted site (i.e., adjacent to the targeted site). In other aspects, the upstream sequence shares substantial sequence identity with a chromosomal sequence that is located within about one hundred (100) nucleotides upstream from the targeted site. Thus, for example, the upstream sequence can share substantial sequence identity with a chromosomal sequence that is located about 1 to about 20, about 21 to about 40, about 41 to about 60, about 61 to about 80, or about 81 to about 100 nucleotides upstream from the targeted site. In one embodiment, the downstream sequence shares substantial sequence identity with a chromosomal sequence located immediately downstream of the targeted site (i.e., adjacent to the targeted site). In other aspects, the downstream sequence shares substantial sequence identity with a chromosomal sequence that is located within about one hundred (100) nucleotides downstream from the targeted site. Thus, for example, the downstream sequence can share substantial sequence identity with a chromosomal sequence that is located about 1 to about 20, about 21 to about 40, about 41 to about 60, about 61 to about 80, or about 81 to about 100 nucleotides downstream from the targeted site. Each upstream or downstream sequence can range in length from about 10 nucleotides to about 5000 nucleotides. In some aspects, upstream and downstream sequences can comprise about 10 to about 50, from about 50 to about 100, from about 100 to about 500, from about 500 to about 1000, from about 1000 to about 2000, or from about 2000 to about nucleotides. In certain aspects, upstream and downstream sequences can range in length from about 20 to about 500 nucleotides.

In still other aspects, the epigenetically modified nucleic acid can be flanked (upstream, downstream, or both) by at least one sequence that is recognized (and cleaved) by a targeting endonuclease. In specific aspects, the epigenetically modified nucleic acid can be flanked on both sides by a target site recognized by a targeting endonuclease. In such instances, the targeting endonuclease also can cleave a larger polynucleotide comprising the epigenetically modified nucleic acid, thereby releasing the epigenetically modified nucleic acid as a linear molecule with overhangs compatible with overhangs in the chromosomal DNA generated by the targeting endonuclease. As a consequence, the released sequence comprising the epigenetically modified nucleic acid can be inserted into the desired chromosomal location by direct ligation. Accordingly, the ends of the sequences to be ligated can be blunt or sticky ends.

In embodiments in which the epigenetically modified nucleic acid is flanked by at least one additional sequence as detailed above, the epigenetically modified nucleic acid can be part of a larger polynucleotide. In some instances, the larger polynucleotide comprising the epigenetically modified nucleic acid and the additional sequence(s) can be linear. In other instances the polynucleotide comprising the epigenetically modified nucleic acid and the additional sequence(s) can be circular. For example, it may be part of a vector.

In embodiments in which the epigenetically modified nucleic acid is part of a vector, a variety of vectors can be used. Suitable vectors include, without limit, plasmid vectors, phagemids, cosmids, artificial/mini-chromosomes, transposons, and viral vectors. In one embodiment, the epigenetically modified nucleic acid is present in a plasmid vector. Non-limiting examples of suitable plasmid vectors include pUC, pBR322, pET, pBluescript, and variants thereof. The vector can comprise additional sequences such as origins of replication, selectable marker sequences (e.g., antibiotic resistance genes), and the like. Additional information can be found in “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001.

In some embodiments, the vector comprising the epigenetically modified nucleic acid can further comprise sequence encoding a marker protein. In one aspect the marker protein is a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1,), blue fluorescent proteins (e.g. EBFP, EBFP2, Azurite, mKalama1, GFPuv, Sapphire, T-sapphire,), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato).

(d) Production of Epigenetically Modified Nucleic Acids

The epigenetically modified nucleic acids can be synthesized using conventional phosphoramidite solid phase oligonucleotide synthesis techniques, but in which standard cytosine phosphoramidites are replaced at the appropriate positions with modified cytosine phosphoramidites. Modified cytosine phosphoramidites such as 5-methylcytosine phosphoramidite, 5-hydroxymethylcytosine phosphoramidite, 5-formylcytosine phosphoramidite, 5-carboxtcytosine phosphoramidite, 3-methylcytosine phosphoramidite, etc. are commercially available. Those of skill in the art are familiar with suitable means for modifying the standard synthesis and deprotection steps when using modified cytosine phosphoramidites.

### II. Cells Comprising Nucleic Acids Having Epigenetic Modifications

The present disclosure also provides genetically engineered cells or cell lines comprising at least one synthetic nucleic acid having predetermined epigenetic modification, as detailed above in section I. In general, the genetically engineered cells or cell lines comprise at least one chromosomally integrated, epigenetically modified nucleic acid, wherein the epigenetic modification is correlated with a known diagnosis, prognosis, or level of sensitivity to a disease treatment. Cells or cell lines comprising chromosomally integrated, epigenetically modified nucleic acid(s) may by prepared by any method known to one of ordinary skill in the art. The epigenetic modification is preferably stable, such that cells or cell lines may be reliably used for any of the uses described herein, for example to control gene expression, serve as reference standards in diagnostic and prognostic assays, and/or assess treatment regimens. Stable modification is desirably maintained throughout cell growth and culture, and cells comprising chromosomally integrated nucleic acids with stable epigenetic modification may be prepared as cell lines using techniques known to one of ordinary skill in the art. However, in some aspects, the epigenetic modification may be metastable. Cells harboring metastable modification may be used to analyze the epigenetic stability with precision based on a priori knowledge of the epigenetic modification pattern or status in the endogenous chromosomal sequence corresponding to the epigenetically modified nucleic acid. The genome of the cell may be modified to include nucleic acids with predetermined modifications using targeting endonuclease-mediated genome editing as described infra.

In the cells or cell lines disclosed herein, the epigenetically modified nuclei acid cam be inserted at the locus of a corresponding endogenous chromosomal sequence having an unmodified or native epigenetic status, wherein the endogenous chromosomal sequence has been deleted or inactivated. For example, the epigenetically modified nucleic acid can be exchanged with the homologous endogenous chromosomal sequence from which the epigenetically modified nucleic acid was derived. Alternatively, the epigenetically modified nucleic acid can be inserted at a locus in which the epigenetic modification is stable, such as a locus possessing adjacent insulating elements, for example genomic safe harbors such as AAVS1, ROSA26, HPRT, and CCR5 loci. In this embodiment, the endogenous chromosomal sequence corresponding to the epigenetically modified synthetic sequence can be optionally inactivated or deleted. As discussed in further detail herein, the epigenetically modified synthetic nucleic acids have substantial sequence identity with regulatory sequences (i.e., control elements) or coding sequences of genes of interest.

(a) Cell Types

Cells contemplated in the present disclosure can and will vary. In general, the cell is a eukaryotic cell. In various aspects, the cell may be a human cell, a non-human mammalian cell, a non-mammalian vertebrate cell, an invertebrate cell, an insect cell, a plant cell, a yeast cell, or a single cell eukaryotic organism. The cell may be an adult cell or an embryonic cell (e.g., an embryo). In still other aspects, the cell may be a stem cell. Suitable stem cells include without limit embryonic stem cells, ES-like stem cells, fetal stem cells, adult stem cells, pluripotent stem cells, induced pluripotent stem cells, multipotent stem cells, oligopotent stem cells, unipotent stem cells and others. In exemplary aspects, the cell is a mammalian cell.

In some aspects, the cell is a cell line cell. Non-limiting examples of suitable mammalian cells include Chinese hamster ovary (CHO) cells, baby hamster kidney (BHK) cells; mouse myeloma NS0 cells, mouse embryonic fibroblast 3T3 cells (NIH3T3), mouse B lymphoma A20 cells; mouse melanoma B16 cells; mouse myoblast C2C12 cells; mouse myeloma SP2/0 cells; mouse embryonic mesenchymal C3H-10T1/2 cells; mouse carcinoma CT26 cells, mouse prostate DuCuP cells; mouse breast EMT6 cells; mouse hepatoma Hepa1c1c7 cells; mouse myeloma J5582 cells; mouse epithelial MTD-1A cells; mouse myocardial MyEnd cells; mouse renal RenCa cells; mouse pancreatic RIN-5F cells; mouse melanoma X64 cells; mouse lymphoma YAC-1 cells; rat glioblastoma 9L cells; rat B lymphoma RBL cells; rat neuroblastoma B35 cells; rat hepatoma cells (HTC); buffalo rat liver BRL 3A cells; canine kidney cells (MDCK); canine mammary (CMT) cells; rat osteosarcoma D17 cells; rat monocyte/macrophage DH82 cells; monkey kidney SV-40 transformed fibroblast (COS7) cells; monkey kidney CVI-76 cells; African green monkey kidney (VERO-76) cells; human embryonic kidney cells (HEK293, HEK293T); human cervical carcinoma cells (HELA); human lung cells (W138); human liver cells (Hep G2); human U2-OS osteosarcoma cells, human A549 cells, human A-431 cells, human SW48 cells, human HCT116 cells, and human K562 cells. An extensive list of mammalian cell lines may be found in the American Type Culture Collection catalog (ATCC, Manassas, Va.). In specific embodiments, the cell is a human cell line cell.

(b) Optional Nucleic Acids

In some aspects, cells comprising the epigenetically modified nucleic acids disclosed herein further can comprise at least one nucleic acid sequence encoding a recombinant protein. The nucleic acid encoding a recombinant protein can be located in the chromosomal of the cell or it can be extrachromosomal. In general, the encoded recombinant protein is heterologous, meaning that the protein is not native to the cell. In some aspects, the recombinant protein may be a therapeutic protein. An exemplary recombinant therapeutic protein includes, without limit, an antibody, a fragment of an antibody, a monoclonal antibody, a humanized antibody, a humanized monoclonal antibody, a chimeric antibody, an IgG molecule, an IgG heavy chain, an IgG light chain, an IgA molecule, an IgD molecule, an IgE molecule, an IgM molecule, a vaccine, a growth factor, a cytokine, an interferon, an interleukin, a hormone, a clotting (or coagulation) factor, a blood component, an enzyme, a nutraceutical protein, a functional fragment or functional variant of any of the forgoing, or a fusion protein comprising any of the foregoing proteins and/or functional fragments or variants thereof.

In other aspects, the recombinant protein may be a protein that imparts improved properties to the cell or improved properties to a first recombinant protein. Non-limiting examples of improved properties include increased robustness, increased viability, increased survival, increased proliferation, increased cell cycle progression (i.e., increased progression from G1 to S phase), increased cell growth, increased cell size, increased production of endogenous proteins, increased production of heterologous proteins, increased stability of a recombinant protein, altered post-translational processing of a recombinant protein, and combinations of any of the above. In some aspects, the protein that improves cell properties may be overexpressed. Non-limiting examples of suitable proteins include serpin proteins (e.g., SerpinB1), cell regulatory proteins, cell cycle control proteins, apoptotic inhibitors, metabolic pathway proteins, post-translation modification proteins, artificial transcription factors, transcriptional activators, transcriptional inhibitors, and enhancer proteins.

In further embodiments, the recombinant protein can be a marker protein, such as a fluorescent protein (examples of which are detailed above), or a selectable marker protein, such as hypoxanthine-guanine phosphoribosyltransferase (HPRT), dihydrofolate reductase (DHFR), and/or glutamine synthase (GS), or a protein encoded by an antibiotic resistance gene.

### III. Generation of Cells Comprising Chromosomally Integrated Nucleic Acids Having Predetermined Epigenetic Modification Using Targeted Endonucleases

Another aspect of the present disclosure provides methods for preparing the cells detailed above in section II. The methods comprise inserting into the genome of a cell a synthetic nucleic acid having a predetermined epigenetic modification(s), and optionally disabling (inactivating) or deleting the corresponding endogenous sequence. The epigenetically modified nucleic acid can have a cytosine modification(s), such as methylation (including 5-methylcytosine (5mC), 3-methylcytosine (3mC), and 5-hydroxymethylcytosine), 5-formylcytosine (5fC) and 5-carboxylcytosine (5caC). In specific aspects, the modification is cytosine methylation. The synthetic nucleic acid can be hypermethylated or hypomethylated as compared with the level of methylation found in the corresponding endogenous sequence of normal cells or cells having a particular phenotype, or the level of methylation found in sequence corresponding to normal level of gene expression or abnormal level of gene expression (i.e., over- or under-expression).

The epigenetically modified nucleic acid can be inserted at the locus of the corresponding endogenous sequence or can be inserted at a different locus, for example, a locus that confers stability to the epigenetically modified nucleic acid.

The epigenetically modified nucleic acid can for example replace the corresponding endogenous chromosomal sequence outright. Such replacement (deletion of the endogenous sequence and insertion of the synthetic epigenetically modified sequence) may be accomplished using methods known in the art, such as the use of targeted endonucleases. Alternatively, the epigenetically modified nucleic acid can be inserted at a favorable locus within the genome, such as a locus possessing adjacent insulating elements or other genetic elements which help maintain the epigenetic modification status (or pattern) of the epigenetically modified nucleic acid prior to chromosomal integration. Loci possessing stabilizing influences are known as genomic safe harbor sites and include loci such as AAVS1, CCR5, HPRT, and ROSA26. Exogenous insulating elements may also be placed in proximity to the epigenetically modified nucleic acid to assist in maintaining the desired modification state. Thus, in one aspect, both the epigenetically modified nucleic acid and insulating elements can be placed at the locus of the corresponding endogenous chromosomal sequence. As mentioned above, targeting endonucleases can be used to integrate the epigenetically modified nucleic acid into the genomic loci of interest.

Any suitable targeting endonuclease may be used to insert the epigenetically modified nucleic acid at the locus of the corresponding endogenous sequence or other favorable locus. For example, the targeting endonuclease can be a zinc finger nuclease, a CRISPR-based endonuclease, a meganuclease, a transcription activator-like effector nuclease (TALEN), an I-TevI nuclease or related monomeric hybrid, or an artificial targeted DNA double strand break inducing agent. For example, paired zinc finger nucleases accomplish non-homologous end-joining (NHEJ) while simultaneously inserting the epigenetically modified nucleic acid of interest. See, e.g., Orlando et al., Nuc. Acids Res., 38(15): e152 (2010). Alternatively, modified RNA-guided endonucleases or transcription activator-like effector nucleases (TALENs) may be used. TALENs generated using the catalytic domain of I-TevI may be prepared and used as described in Beurdeley et al., Nat. Commun. 4: 1762 doi: 10.1038/ncomms2782 (2013). One of ordinary skill in the art will understand that hybrid endonucleases may also be used, such as an I-Tev nuclease domain fused to zinc finger endonucleases or LAGLIDADG homing endonuclease scaffolds, as described in Kleinstiver et al., PNAS 109(21): 8061-6 (2012). An artificial targeted DNA double strand break inducing agent may also be used to promote homologous recombination in the present methods, such as an ARCUT (Artificial Restriction DNA Cutter) as described in Katada et al., Nuc. Acid Res. 40(11): e81 (2012).

The present disclosure encompasses a method for inserting a synthetic nucleic acid having a predetermined epigenetic modification into a eukaryotic cell using a targeting endonuclease, such as any of the targeting endonucleases described herein. The method comprises introducing into a cell (i) at least one targeting endonuclease or nucleic acid(s) encoding the at least one targeting endonuclease, wherein each targeting endonuclease is targeted to a site in the cell's endogenous chromosomal sequence, and (ii) at least one synthetic nucleic acid having a predetermined epigenetic modification. In some aspects, the epigenetically modified nucleic acid may be a linear sequence comprising overhangs compatible with those generated by the targeting endonuclease. In other aspects, the epigenetically modified nucleic acid can be flanked by upstream and downstream sequences that have substantial sequence identity with sequences on either side of the targeted cleavage site in the cell's genome. In further aspects, the epigenetically modified nucleic acid can be flanked by target sites that are recognized by the targeting endonuclease. The method further comprises culturing the cell such that the targeting endonuclease(s) introduces at least one double-stranded break, which is repaired by a DNA repair process that leads to insertion of the epigenetically modified nucleic acid into a targeted site and/or inactivation of the endogenous chromosomal sequence at a targeted site. For example, a targeting endonuclease can be used to create one double-stranded break at the targeted locus, wherein the epigenetically modified nucleic acid comprising compatible overhangs is ligated with the endogenous chromosomal sequence thereby inserting the epigenetically modified nucleic acid at the targeted locus and disrupting/inactivating the endogenous chromosomal sequence. The targeted locus can correspond to the endogenous chromosomal sequence from which the epigenetically modified nucleic acid is derived or the targeted locus can be a genomic safe harbor site. Alternatively, a targeting endonuclease can be used to create one double-stranded break, wherein the epigenetically modified nucleic acid comprising homologous upstream and downstream sequences is inserted into the cleavage site by a homology-directed repair process. In another aspect, two targeting endonucleases can be used to create two double-stranded breaks at targeted sites within the locus of interest, wherein the epigenetically modified nucleic acid is exchanged with the endogenous chromosomal sequence during repair of the double-stranded breaks. In still another aspect, a first targeting endonuclease can be used to create a double-stranded break at a first locus in which the epigenetically modified nucleic acid is inserted, and a second targeting endonuclease can be used to create a double-stranded break at a second locus, which break is repaired by an error-prone DNA repair process such that at an inactivating mutation is introduced at the second locus. For example, the first locus can be a site that confers stability to the epigenetically modified nucleic acid, and the second locus can correspond to the endogenous chromosomal sequence from which the epigenetically modified nucleic acid was derived.

(a) Targeting Endonucleases

The type of targeting endonuclease used in the method disclosed herein can and will vary. As mentioned above, the targeting endonuclease can be a meganuclease, a transcription activator-like effector nuclease (TALEN), a I-TevI nuclease or related monomeric hybrid, and an artificial targeted DNA double strand break inducing agent, a zinc finger nuclease (ZFN), or a CRISPR-based endonuclease. The targeting endonuclease can be a naturally-occurring protein or an engineered protein.

In some aspects, the targeting endonuclease can be a meganuclease. Meganucleases are endodeoxyribonucleases characterized by a large recognition site, i.e., the recognition site generally ranges from about 12 base pairs to about 40 base pairs. As a consequence of this requirement, the recognition site generally occurs only once in any given genome. Among meganucleases, the family of homing endonucleases named LAGLIDADG has become a valuable tool for the study of genomes and genome engineering (Chevalier et al., Nuc Acids Mol. Biol. 16:33-27 (2005)). Meganucleases can be targeted to specific chromosomal sequences by modifying their recognition sequence using techniques well known to those skilled in the art. See, e.g., Silva et al., Curr. Gene Ther. 11(1):11-27 (2011); Baxter et al., Nuc. Acids Res. 40(160:7985-8000 (22012).

In other aspects, the targeting endonuclease can be a transcription activator-like effector (TALE) nuclease. TALEs are transcription factors from the plant pathogen Xanthomonas that may be readily engineered to bind new DNA targets. TALEs or truncated versions thereof may be linked to the catalytic domain of endonucleases such as FokI to create targeting endonuclease called TALE nucleases or TALENs. For additional information, see Joung et al., Nature Rev. Mol. Cell Biol. 14:49-55 (2013). TALENs generated using the catalytic domain of I-TevI may be prepared and used as described in Beurdeley et al., Nat. Commun., 4: 1762 doi: 10.1038/ncomms2782 (2013).

In additional aspects, the targeting endonuclease can be an I-TevI nuclease or related monomeric hybrid, such as an I-Tev nuclease domain fused to zinc finger endonucleases or LAGLIDADG homing endonuclease scaffolds, as described in Kleinstiver et al., PNAS, 109(21): 8061-6 (2012).

In still other aspects, the targeting nuclease can be an artificial targeted DNA double strand break inducing agent. An artificial targeted DNA double strand break inducing agent can be used to promote homologous recombination in the present methods, such as an ARCUT (Artificial Restriction DNA Cutter) as described in Katada et al., Nuc. Acid Res. 40(11): e81 (2012).

(i) Zinc Finger Nuclease

In further aspects, the targeting endonuclease can be a zinc finger nuclease (ZFN). Typically, a zinc finger nuclease comprises a DNA binding domain (i.e., zinc finger) and a cleavage domain (i.e., nuclease), both of which are described below.

Zinc Finger Binding Domain.

Zinc finger binding domains may be engineered to recognize and bind to any nucleic acid sequence of choice. See, for example, Beerli et al. (2002) Nat. Biotechnol. 20:135-141; Pabo et al. (2001) Ann. Rev. Biochem. 70:313-340; Isalan et al. (2001) Nat. Biotechnol. 19:656-660; Segal et al. (2001) Curr. Opin. Biotechnol. 12:632-637; Choo et al. (2000) Curr. Opin. Struct. Biol. 10:411-416; Zhang et al. (2000) J. Biol. Chem. 275(43):33850-33860; Doyon et al. (2008) Nat. Biotechnol. 26:702-708; and Santiago et al. (2008) Proc. Natl. Acad. Sci. USA 105:5809-5814. An engineered zinc finger binding domain can have a novel binding specificity compared to a naturally-occurring zinc finger protein. Engineering methods include, but are not limited to, rational design and various types of selection. Rational design includes, for example, using databases comprising doublet, triplet, and/or quadruplet nucleotide sequences and individual zinc finger amino acid sequences, in which each doublet, triplet or quadruplet nucleotide sequence is associated with one or more amino acid sequences of zinc fingers which bind the particular triplet or quadruplet sequence. See, for example, U.S. Pat. Nos. 6,453,242 and 6,534,261, the disclosures of which are incorporated by reference herein in their entireties. As an example, the algorithm described in U.S. Pat. No. 6,453,242 may be used to design a zinc finger binding domain to target a preselected sequence. Alternative methods, such as rational design using a nondegenerate recognition code table can also be used to design a zinc finger binding domain to target a specific sequence (Sera et al. (2002) Biochemistry 41:7074-7081). Publically available web-based tools for identifying potential target sites in DNA sequences and designing zinc finger binding domains are found at www.zincfingertools.org and zifit.partners.org/ZiFiT/, respectively (Mandell et al. (2006) Nuc. Acid Res. 34:W516-W523; Sander et al. (2007) Nuc. Acid Res. 35:W599-W605).

A zinc finger binding domain may be designed to recognize and bind a DNA sequence ranging from about 3 nucleotides to about 21 nucleotides in length, for example, from about 9 to about 18 nucleotides in length. Each zinc finger recognition region (i.e., zinc finger) recognizes and binds three nucleotides. In general, the zinc finger binding domains of the zinc finger nucleases disclosed herein comprise at least three zinc finger recognition regions (i.e., zinc fingers). The zinc finger binding domain may for example comprise four zinc finger recognition regions. Alternatively, the zinc finger binding domain may comprise five or six zinc finger recognition regions. A zinc finger binding domain may be designed to bind to any suitable target DNA sequence. See for example, U.S. Pat. Nos. 6,607,882; 6,534,261 and 6,453,242, the disclosures of which are incorporated by reference herein in their entireties.

Exemplary methods of selecting a zinc finger recognition region include phage display and two-hybrid systems, and are disclosed in U.S. Pat. Nos. 5,789,538; 5,925,523; 6,007,988; 6,013,453; 6,410,248; 6,140,466; 6,200,759; and 6,242,568; as well as WO 98/37186; WO 98/53057; WO 00/27878; WO 01/88197 and GB 2,338,237, each of which is incorporated by reference herein in its entirety. In addition, enhancement of binding specificity for zinc finger binding domains has been described, for example, in WO 02/077227, the disclosure of which is incorporated herein by reference.

Zinc finger binding domains and methods for design and construction of fusion proteins (and polynucleotides encoding same) are known to those of skill in the art and are described in detail in U.S. Patent Application Publication Nos. 20050064474 and 20060188987, each incorporated by reference herein in its entirety. Zinc finger recognition regions and/or multi-fingered zinc finger proteins may be linked together using suitable linker sequences, including for example, linkers of five or more amino acids in length. See, U.S. Pat. Nos. 6,479,626; 6,903,185; and 7,153,949, the disclosures of which are incorporated by reference herein in their entireties, for non-limiting examples of linker sequences of six or more amino acids in length. The zinc finger binding domain described herein may include a combination of suitable linkers between the individual zinc fingers (and additional domains) of the protein.

Cleavage Domain.

A zinc finger nuclease also includes a cleavage domain. The cleavage domain portion of the zinc finger nuclease may be obtained from any endonuclease or exonuclease. Non-limiting examples of endonucleases from which a cleavage domain may be derived include, but are not limited to, restriction endonucleases and homing endonucleases. See, for example, New England Biolabs catalog (www.neb.com) and Belfort et al. (1997) Nucleic Acids Res. 25:3379-3388. Additional enzymes that cleave DNA are known (e.g., S1 Nuclease; mung bean nuclease; pancreatic DNase I; micrococcal nuclease; yeast HO endonuclease). See also Linn et al. (eds.) Nucleases, Cold Spring Harbor Laboratory Press, 1993. One or more of these enzymes (or functional fragments thereof) may be used as a source of cleavage domains.

A cleavage domain also may be derived from an enzyme or portion thereof, as described above, that requires dimerization for cleavage activity. Two zinc finger nucleases may be required for cleavage, as each nuclease comprises a monomer of the active enzyme dimer. Alternatively, a single zinc finger nuclease can comprise both monomers to create an active enzyme dimer. As used herein, an “active enzyme dimer” is an enzyme dimer capable of cleaving a nucleic acid molecule. The two cleavage monomers may be derived from the same endonuclease (or functional fragments thereof), or each monomer may be derived from a different endonuclease (or functional fragments thereof).

When two cleavage monomers are used to form an active enzyme dimer, the recognition sites for the two zinc finger nucleases are preferably disposed such that binding of the two zinc finger nucleases to their respective recognition sites places the cleavage monomers in a spatial orientation to each other that allows the cleavage monomers to form an active enzyme dimer, e.g., by dimerizing. As a result, the near edges of the recognition sites may be separated by about 5 to about 18 nucleotides. For instance, the near edges may be separated by about 5, 6, 7, 8, 9, 10, 11, 12, 13, 14, 15, 16, 17 or 18 nucleotides. It will however be understood that any integral number of nucleotides or nucleotide pairs can intervene between two recognition sites (e.g., from about 2 to about 50 nucleotide pairs or more). The near edges of the recognition sites of the zinc finger nucleases, such as for example those described in detail herein, may be separated by 6 nucleotides. In general, the site of cleavage lies between the recognition sites.

Restriction endonucleases (restriction enzymes) are present in many species and are capable of sequence-specific binding to DNA (at a recognition site), and cleaving DNA at or near the site of binding. Certain restriction enzymes (e.g., Type IIS) cleave DNA at sites removed from the recognition site and have separable binding and cleavage domains. For example, the Type IIS enzyme FokI catalyzes double-stranded cleavage of DNA, at 9 nucleotides from its recognition site on one strand and 13 nucleotides from its recognition site on the other. See, for example, U.S. Pat. Nos. 5,356,802; 5,436,150 and 5,487,994; as well as Li et al. (1992) Proc. Natl. Acad. Sci. USA 89:4275-4279; Li et al. (1993) Proc. Natl. Acad. Sci. USA 90:2764-2768; Kim et al. (1994a) Proc. Natl. Acad. Sci. USA 91:883-887; Kim et al. (1994b) J. Biol. Chem. 269:31, 978-31, 982. Thus, a zinc finger nuclease can comprise the cleavage domain from at least one Type IIS restriction enzyme and one or more zinc finger binding domains, which may or may not be engineered. Exemplary Type IIS restriction enzymes are described for example in International Publication WO 07/014,275, the disclosure of which is incorporated by reference herein in its entirety. Additional restriction enzymes also contain separable binding and cleavage domains, and these also are contemplated by the present disclosure. See, for example, Roberts et al. (2003) Nucleic Acids Res. 31:418-420.

An exemplary Type IIS restriction enzyme, whose cleavage domain is separable from the binding domain, is FokI. This particular enzyme is active as a dimer (Bitinaite et al. (1998) Proc. Natl. Acad. Sci. USA 95: 10, 570-10, 575). Accordingly, for the purposes of the present disclosure, the portion of the FokI enzyme used in a zinc finger nuclease is considered a cleavage monomer. Thus, for targeted double-stranded cleavage using a FokI cleavage domain, two zinc finger nucleases, each comprising a FokI cleavage monomer, may be used to reconstitute an active enzyme dimer. Alternatively, a single polypeptide molecule containing a zinc finger binding domain and two FokI cleavage monomers can also be used.

The cleavage domain may comprise one or more engineered cleavage monomers that minimize or prevent homodimerization, as described, for example, in U.S. Patent Publication Nos. 20050064474, 20060188987, and 20080131962, each of which is incorporated by reference herein in its entirety. By way of non-limiting example, amino acid residues at positions 446, 447, 479, 483, 484, 486, 487, 490, 491, 496, 498, 499, 500, 531, 534, 537, and 538 of FokI are all targets for influencing dimerization of the FokI cleavage half-domains. Exemplary engineered cleavage monomers of FokI that form obligate heterodimers include a pair in which a first cleavage monomer includes mutations at amino acid residue positions 490 and 538 of FokI and a second cleavage monomer that includes mutations at amino-acid residue positions 486 and 499 (Miller et al., 2007, Nat. Biotechnol, 25:778-785; Szczpek et al., 2007, Nat. Biotechnol, 25:786-793). For example, the Glu (E) at position 490 may be changed to Lys (K) and the lie (I) at position 538 may be changed to K in one domain (E490K, 1538K), and the Gin (Q) at position 486 may be changed to E and the I at position 499 may be changed to Leu (L) in another cleavage domain (Q486E, 1499L). In other aspects, modified FokI cleavage domains can include three amino acid changes (Doyon et al. 2011, Nat. Methods, 8:74-81). For example, one modified FokI domain (which is termed ELD) can comprise Q486E, 1499L, N496D mutations and the other modified FokI domain (which is termed KKR) can comprise E490K, 1538K, H537R mutations.

Additional Domains.

In some aspects, the zinc finger nuclease further comprises at least one nuclear localization signal or sequence (NLS). A NLS is an amino acid sequence that facilitates transport of the zinc finger nuclease protein into the nucleus of eukaryotic cells. In general, an NLS comprise a stretch of basic amino acids. Nuclear localization signals are known in the art (see, e.g., Makkerh et al., 1996, Current Biology 6:1025-1027; Lange et al., J. Biol. Chem., 2007, 282:5101-5105). For example, in one embodiment, the NLS can be a monopartite sequence, such as PKKKRKV (SEQ ID NO:1) or PKKKRRV (SEQ ID NO:2). In another embodiment, the NLS can be a bipartite sequence. In still another embodiment, the NLS can be KRPAATKKAGQAKKKK (SEQ ID NO:3). The NLS can be located at the N-terminus, the C-terminus, or in an internal location of the zinc finger nuclease.

In other aspects, the zinc finger nuclease can also comprise at least one cell-penetrating domain. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO:4). In another embodiment, the cell-penetrating domain can be TLM (PLSSIFSRIGDPPKKKRKV; SEQ ID NO:5), a cell-penetrating peptide sequence derived from the human hepatitis B virus. In still another embodiment, the cell-penetrating domain can be MPG (GALFLGWLGAAGSTMGAPKKKRKV; SEQ ID NO:6 or GALFLGFLGAAGSTMGAWSQPKKKRKV; SEQ ID NO:7). In an additional embodiment, the cell-penetrating domain can be Pep-1 (KETWWETWWTEWSQPKKKRKV; SEQ ID NO:8), VP22, a cell penetrating peptide from Herpes simplex virus, or a polyarginine peptide sequence. The cell-penetrating domain can be located at the N-terminus, the C-terminus, or in an internal location of the protein.

In still other aspects, the zinc finger nuclease can further comprise at least one marker domain. Non-limiting examples of marker domains include fluorescent proteins, purification tags, and epitope tags. In one embodiment, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1,), blue fluorescent proteins (e.g. EBFP, EBFP2, Azurite, mKalama1, GFPuv, Sapphire, T-sapphire,), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In another embodiment, the marker domain can be a purification tag and/or an epitope tag. Suitable tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin. The marker domain can be located at the N-terminus, the C-terminus, or in an internal location of the zinc finger nuclease protein.

(ii) CRISPR-Based Endonucleases

In still other aspects, the targeting endonuclease can be a CRISPR-based endonuclease comprising at least one nuclear localization signal, which permits entry of the endonuclease into the nuclei of eukaryotic cells. CRISPR-based endonucleases are RNA-guided endonucleases that comprise at least one nuclease domain and at least one domain that interacts with a guide RNA. A guide RNA directs the CRISPR-based endonucleases to a targeted site in a nucleic acid at which site the CRISPR-based endonucleases cleaves at least one strand of the targeted nucleic acid sequence. Since the guide RNA provides the specificity for the targeted cleavage, the CRISPR-based endonuclease is universal and may be used with different guide RNAs to cleave different target nucleic acid sequences.

CRISPR-based endonucleases are RNA-guided endonucleases derived from CRISPR/Cas systems. Bacteria and archaea have evolved an RNA-based adaptive immune system that uses CRISPR (clustered regularly interspersed short palindromic repeat) and Cas (CRISPR-associated) proteins to detect and destroy invading viruses or plasmids. CRISPR/Cas endonucleases can be programmed to introduce targeted site-specific double-strand breaks by providing target-specific synthetic guide RNAs (Jinek et al., 2012, Science, 337:816-821).

The CRISPR-based endonuclease can be derived from a CRISPR/Cas type I, type II, or type III system. Non-limiting examples of suitable CRISPR/Cas proteins include Cas3, Cas4, Cas5, Cas5e (or CasD), Cas6, Cas6e, Cas6f, Cas7, Cas8a1, Cas8a2, Cas8b, Cas8c, Cas9, Cas10, Cas10d, CasF, CasG, CasH, Csy1, Csy2, Csy3, Cse1 (or CasA), Cse2 (or CasB), Cse3 (or CasE), Cse4 (or CasC), Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csz1, Csx15, Csf1, Csf2, Csf3, Csf4, and Cu1966.

In one embodiment, the CRISPR-based endonuclease is derived from a type II CRISPR/Cas system. In exemplary embodiments, the CRISPR-based endonuclease is derived from a Cas9 protein. The Cas9 protein can be from Streptococcus pyogenes, Streptococcus thermophilus, Streptococcus sp., Nocardiopsis dassonvillei, Streptomyces pristinaespiralis, Streptomyces viridochromogenes, Streptomyces viridochromogenes, Streptosporangium roseum, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microscilla marina, Burkholderiales bacterium, Polaromonas naphthalenivorans, Polaromonas sp., Crocosphaera watsonii, Cyanothece sp., Microcystis aeruginosa, Synechococcus sp., Acetohalobium arabaticum, Ammonifex degensii, Caldicelulosiruptor becscii, Candidatus Desulforudis, Clostridium botulinum, Clostridium difficile, Finegoldia magna, Natranaerobius thermophilus, Pelotomaculum thermopropionicum, Acidithiobacillus caldus, Acidithiobacillus ferrooxidans, Allochromatium vinosum, Marinobacter sp., Nitrosococcus halophilus, Nitrosococcus watsoni, Pseudoalteromonas haloplanktis, Ktedonobacter racemifer, Methanohalobium evestigatum, Anabaena variabilis, Nodularia spumigena, Nostoc sp., Arthrospira maxima, Arthrospira platensis, Arthrospira sp., Lyngbya sp., Microcoleus chthonoplastes, Oscillatoria sp., Petrotoga mobilis, Thermosipho africanus, or Acaryochloris marina. In one specific embodiment, the CRISPR-based nuclease is derived from a Cas9 protein from Streptococcus pyogenes.

In general, CRISPR/Cas proteins comprise at least one RNA recognition and/or RNA binding domain. RNA recognition and/or RNA binding domains interact with the guide RNA such that the CRISPR/Cas protein is directed to a specific genomic or genomic sequence. CRISPR/Cas proteins can also comprise nuclease domains (i.e., DNase or RNase domains), DNA binding domains, helicase domains, protein-protein interaction domains, dimerization domains, as well as other domains.

The CRISPR-based endonuclease used herein can be a wild type CRISPR/Cas protein, a modified CRISPR/Cas protein, or a fragment of a wild type or modified CRISPR/Cas protein. The CRISPR/Cas protein can be modified to increase nucleic acid binding affinity and/or specificity, alter an enzymatic activity, and/or change another property of the protein. For example, nuclease (i.e., DNase, RNase) domains of the CRISPR/Cas protein can be modified, deleted, or inactivated. The CRISPR/Cas protein can be truncated to remove domains that are not essential for the function of the protein. The CRISPR/Cas protein also can be truncated or modified to optimize the activity of the protein or an effector domain fused with the CRISPR/Cas protein.

In some embodiments, the CRISPR-based endonuclease can be derived from a wild type Cas9 protein or fragment thereof. In other embodiments, the CRISPR-based endonuclease can be derived from a modified Cas9 protein. For example, the amino acid sequence of the Cas9 protein can be modified to alter one or more properties (e.g., nuclease activity, affinity, stability, etc.) of the protein. Alternatively, domains of the Cas9 protein not involved in RNA-guided cleavage can be eliminated from the protein such that the modified Cas9 protein is smaller than the wild type Cas9 protein.

In general, a Cas9 protein comprises at least two nuclease (i.e., DNase) domains. For example, a Cas9 protein can comprise a RuvC-like nuclease domain and a HNH-like nuclease domain. The RuvC and HNH domains work together to cut single strands to make a double-strand break in DNA (Jinek et al., 2012, Science, 337:816-821). In one embodiment, the CRISPR-based endonuclease is derived from a Cas9 protein and comprises two function nuclease domains, which together introduce a double-stranded break into the targeted site.

The target sites recognized by naturally occurring CRISPR/Cas systems typically having lengths of about 14-15 bp (Cong et al., 2013, Science, 339:819-823). The target site has no sequence limitation except that sequence complementary to the 5′ end of the guide RNA (i.e., called a protospacer sequence) is immediately followed by (3′ or downstream) a consensus sequence. This consensus sequence is also known as a protospacer adjacent motif (or PAM). Examples of PAM include, but are not limited to, NGG, NGGNG, and NNAGAAW (wherein N is defined as any nucleotide and W is defined as either A or T). At the typical length, only about 5-7% of the target sites would be unique within a target genome, indicating that off target effects could be significant. The length of the target site can be expanded by requiring two binding events. For example, CRISPR-based endonucleases can be modified such that they can only cleave one strand of a double-stranded sequence (i.e., converted to nickases). Thus, the use of a CRISPR-based nickase in combination with two different guide RNAs would essentially double the length of the target site, while still effecting a double stranded break.

In some embodiments, the Cas9-derived endonuclease can be modified to contain only one functional nuclease domain (either a RuvC-like or a HNH-like nuclease domain). For example, the Cas9-derived protein can be modified such that one of the nuclease domains is deleted or mutated such that it is no longer functional (i.e., the domain lacks nuclease activity). In some embodiments in which one of the nuclease domains is inactive, the Cas9-derived protein is able to introduce a nick into a double-stranded nucleic acid (such protein is termed a “nickase”), but not cleave the double-stranded DNA. For example, an aspartate to alanine (D10A) conversion in a RuvC-like domain converts the Cas9-derived protein into a “HNH” nickase. Likewise, a histidine to alanine (H840A) conversion (in some instances, the histidine is located at position 839) in a HNH domain converts the Cas9-derived protein into a “RuvC” nickase. Thus, for example, in one embodiment the Cas9-derived nickase has an aspartate to alanine (D10A) conversion in a RuvC-like domain. In another embodiment, the Cas9-derived nickase has a histidine to alanine (H840A or H839A) conversion in a HNH domain. The RuvC-like or HNH-like nuclease domains of the Cas9-derived nickase can be modified using well-known methods, such as site-directed mutagenesis, PCR-mediated mutagenesis, and total gene synthesis, as well as other methods known in the art.

In still other embodiments, both nuclease domains of the CRISPR-based endonuclease can be mutated, inactivated, or deleted and the resulting protein can be combined with a heterologous cleavage domain to create a CRISPR-based fusion protein. Thus, the resultant fusion protein is guided to the target site by a guide RNA, and cleavage is mediated by the heterologous cleavage domain. In certain embodiments, the heterologous cleavage domain can be derived from a type II-S endonuclease. Type II-S endonucleases cleave DNA at sites that are typically several base pairs away the recognition site and, as such, have separable recognition and cleavage domains. These enzymes generally are monomers that transiently associate to form dimers to cleave each strand of DNA at staggered locations. Non-limiting examples of suitable type II-S endonucleases include BfiI, BpmI, BsaI, BsgI, BsmBI, BsmI, BspMI, FokI, MboII, and SapI. In exemplary aspects, the cleavage domain of the fusion protein is a FokI cleavage domain or a derivative thereof, which are detailed above in section (III)(a)(i).

In general, the CRISPR-based endonuclease comprises at least one nuclear localization signal or sequence (NLS). Suitable NLS include, without limit, PKKKRKV (SEQ ID NO:1), PKKKRRV (SEQ ID NO:2), and KRPAATKKAGQAKKKK (SEQ ID NO:3). The NLS can be located at the N-terminus, the C-terminus, or in an internal location of the CRISPR-based endonuclease.

Additional Domains.

In other aspects, the CRISPR-based endonuclease can also comprise at least one cell-penetrating domain. In one embodiment, the cell-penetrating domain can be a cell-penetrating peptide sequence derived from the HIV-1 TAT protein. As an example, the TAT cell-penetrating sequence can be GRKKRRQRRRPPQPKKKRKV (SEQ ID NO:4). In another embodiment, the cell-penetrating domain can be TLM (PLSSIFSRIGDPPKKKRKV; SEQ ID NO:5), a cell-penetrating peptide sequence derived from the human hepatitis B virus. In still another embodiment, the cell-penetrating domain can be MPG (GALFLGWLGAAGSTMGAPKKKRKV; SEQ ID NO:6 or GALFLGFLGAAGSTMGAWSQPKKKRKV; SEQ ID NO:7). In an additional embodiment, the cell-penetrating domain can be Pep-1 (KETWWETWWTEWSQPKKKRKV; SEQ ID NO:8), VP22, a cell penetrating peptide from Herpes simplex virus, or a polyarginine peptide sequence. The cell-penetrating domain can be located at the N-terminus, the C-terminus, or in an internal location of the CRISPR-based endonuclease

In still other embodiments, the CRISPR-based endonuclease can comprise at least one marker domain. Non-limiting examples of marker domains include fluorescent proteins, purification tags, and epitope tags. In one embodiment, the marker domain can be a fluorescent protein. Non limiting examples of suitable fluorescent proteins include green fluorescent proteins (e.g., GFP, GFP-2, tagGFP, turboGFP, EGFP, Emerald, Azami Green, Monomeric Azami Green, CopGFP, AceGFP, ZsGreen1), yellow fluorescent proteins (e.g. YFP, EYFP, Citrine, Venus, YPet, PhiYFP, ZsYellow1,), blue fluorescent proteins (e.g. EBFP, EBFP2, Azurite, mKalama1, GFPuv, Sapphire, T-sapphire,), cyan fluorescent proteins (e.g. ECFP, Cerulean, CyPet, AmCyan1, Midoriishi-Cyan), red fluorescent proteins (mKate, mKate2, mPlum, DsRed monomer, mCherry, mRFP1, DsRed-Express, DsRed2, DsRed-Monomer, HcRed-Tandem, HcRed1, AsRed2, eqFP611, mRasberry, mStrawberry, Jred), and orange fluorescent proteins (mOrange, mKO, Kusabira-Orange, Monomeric Kusabira-Orange, mTangerine, tdTomato) or any other suitable fluorescent protein. In another embodiment, the marker domain can be a purification tag and/or an epitope tag. Suitable tags include, but are not limited to, glutathione-S-transferase (GST), chitin binding protein (CBP), maltose binding protein, thioredoxin (TRX), poly(NANP), tandem affinity purification (TAP) tag, myc, AcV5, AU1, AU5, E, ECS, E2, FLAG, HA, nus, Softag 1, Softag 3, Strep, SBP, Glu-Glu, HSV, KT3, S, S1, T7, V5, VSV-G, 6×His, biotin carboxyl carrier protein (BCCP), and calmodulin. The marker domain can be located at the N-terminus, the C-terminus, or in an internal location of the protein.

Guide RNA.

A CRISPR-based endonuclease also requires at least one guide RNA that directs the CRISPR-based endonuclease to a specific target site, at which site the CRISPR-based endonuclease cleaves at least one strand of the targeted sequence. The target site has no sequence limitation except that the sequence is immediately followed (downstream) by a consensus sequence. This consensus sequence is also known as a protospacer adjacent motif (PAM). Examples of PAM include, but are not limited to, NGG, NGGNG, and NNAGAAW (wherein N is defined as any nucleotide and W is defined as either A or T). The target site may be in the coding region of a gene, a promoter control element of a gene, in an intron of a gene, in a control region between genes, etc.

A guide RNA comprises three regions: a first region at the 5′ end that is complementary to the sequence at the target site, a second internal region that forms a stem loop structure, and a third 3′ region that remains essentially single-stranded. The first region of each guide RNA is different such that each guide RNA guides a CRISPR-based endonuclease to a specific target site. The second and third regions of each guide RNA can be the same in all guide RNAs.

The first region of the guide RNA is complementary to sequence at the target site such that the first region of the guide RNA can base pair with sequence at the target site. In various embodiments, the first region of the guide RNA can comprise from about 10 nucleotides to more than about 25 nucleotides. For example, the region of base pairing between the first region of the guide RNA and the target site in the genomic sequence can be about 10, 11, 12, 13, 14, 15, 16, 17, 18, 19, 20, 22, 23, 24, 25, or more than 25 nucleotides in length. In an exemplary embodiment, the first region of the guide RNA is about 20 nucleotides in length.

The guide RNA also comprises a second region that forms a secondary structure. In some embodiments, the secondary structure comprises a stem (or hairpin) and a loop. The length of the loop and the stem can vary. For example, the loop can range from about 3 to about 10 nucleotides in length, and the stem can range from about 6 to about 20 base pairs in length. The stem can comprise one or more bulges of 1 to about 10 nucleotides. Thus, the overall length of the second region can range from about 16 to about 60 nucleotides in length. In an exemplary embodiment, the loop is about 4 nucleotides in length and the stem comprises about 12 base pairs.

The guide RNA also comprises a third region at the 3′ end that remains essentially single-stranded. Thus, the third region has no complementarity to any genomic sequence in the cell of interest and has no complementarity to the rest of the guide RNA. The length of the third region can vary. In general, the third region is more than about 4 nucleotides in length. For example, the length of the third region can range from about 5 to about 30 nucleotides in length.

In some embodiments, the guide RNA comprises one molecule. In other embodiments, the guide RNA can comprise two separate molecules. The first RNA molecule can comprise the first region of the guide RNA and one half of the “stem” of the second region of the guide RNA. The second RNA molecule can comprise the other half of the “stem” of the second region of the guide RNA and the third region of the guide RNA. Thus, in this embodiment, the first and second RNA molecules each contain a sequence of nucleotides that are complementary to one another. For example, in one embodiment, the first and second RNA molecules each comprise a sequence (of about 6 to about 20 nucleotides) that base pairs to the other sequence to form a functional guide RNA.

(b) Delivery of Targeting Endonuclease and Synthetic DNA to the Cell

The method comprises introducing into a cell at least one targeting endonuclease or nucleic acid encoding the at least one targeting endonuclease. Suitable cells are detailed above in section (II)(a). In some aspects, the targeting endonuclease can be introduced into the cell as a purified isolated protein. In such instances, the targeting endonuclease can further comprise at least one cell-penetrating domain. Examples of cell-penetrating domains are detailed above in the sections describing zinc finger nucleases and CRISPR-based endonucleases. The targeting endonuclease can be expressed in and purified from bacterial or eukaryotic cells using techniques well known in the art.

In other aspects, the targeting endonuclease can be introduced into the cell as a nucleic acid. The nucleic acid can be DNA or RNA. In aspects in which the encoding nucleic acid is mRNA, the mRNA may be 5′ capped and/or 3′ polyadenylated. In embodiments in which the targeting endonuclease is a zinc finger nuclease, the encoding nucleic acid can be mRNA. The mRNA coding the zinc finger nuclease can be 5′ capped and 3′ polyadenylated. Methods for preparing RNA, as well as methods for capping and polyadenylating mRNA are known in the art.

In additional aspects, the nucleic acid encoding the targeting endonuclease can be DNA. The DNA may be linear or circular. In certain aspects, the DNA encoding the targeting endonuclease can be part of a vector. Suitable vectors include plasmid vectors, phagemids, cosmids, artificial/mini-chromosomes, transposons, and viral vectors. In a non-limiting example, the DNA encoding the targeting endonuclease is present in a plasmid vector. Non-limiting examples of suitable plasmid vectors include pUC, pBR322, pET, pBluscript, and variants thereof. The DNA encoding the targeting endonuclease generally is operably linked to at least one expression control sequence. In particular, the DNA coding sequence can be operably linked to a promoter control sequence for expression in the cell of interest. The promoter control sequence can be constitutive, regulated, or tissue-specific. Suitable constitutive promoter control sequences include, but are not limited to, cytomegalovirus immediate early promoter (CMV), simian virus (SV40) promoter, adenovirus major late promoter, Rous sarcoma virus (RSV) promoter, mouse mammary tumor virus (MMTV) promoter, phosphoglycerate kinase (PGK) promoter, elongation factor (ED1)-alpha promoter, ubiquitin promoters, actin promoters, tubulin promoters, immunoglobulin promoters, fragments thereof, or combinations of any of the foregoing. Examples of suitable regulated promoter control sequences include without limit those regulated by heat shock, metals, steroids, antibiotics, or alcohol. Non-limiting examples of tissue-specific promoters include B29 promoter, CD14 promoter, CD43 promoter, CD45 promoter, CD68 promoter, desmin promoter, elastase-1 promoter, endoglin promoter, fibronectin promoter, Fit-1 promoter, GFAP promoter, GPIIb promoter, ICAM-2 promoter, INF-β promoter, Mb promoter, NphsI promoter, OG-2 promoter, SP-B promoter, SYN1 promoter, and WASP promoter. The promoter sequence can be wild type or it can be modified for more efficient or efficacious expression. The vector can comprise additional expression control sequences (e.g., enhancer sequences, Kozak sequences, polyadenylation sequences, transcriptional termination sequences, etc.), selectable marker sequences (e.g., antibiotic resistance genes), origins of replication, and the like. Those skilled in the art are familiar with appropriate vectors, promoters, other vector control elements.

In aspects in which the targeting endonuclease is a CRISPR-based endonuclease and the CRISPR-based endonuclease is introduced into the cell as a nucleic acid, the encoding nucleic acid can be codon optimized for efficient translation into protein in the eukaryotic cell of interest. For example, codons can be optimized for expression in humans, mice, rats, hamsters, cows, pigs, cats, dogs, fish, amphibians, plants, yeast, insects, and so forth (see Codon Usage Database at www.kazusa.or.jp/codon). Programs for codon optimization are available as freeware (e.g., OPTIMIZER at genomes.urv.es/OPTIMIZER; OptimumGene™ from GenScript at www.genscript.com/codon_opt.html). Commercial codon optimization programs are also available.

Additionally, when the targeting endonuclease is a CRISPR-based endonuclease, the method further comprises delivering to the cell at least one guide RNA. Generally, the ratio of CRISPR-based endonuclease to guide RNA is about 1:1. In some aspects, the guide RNA can be introduced as an RNA molecule. For example, when the endonuclease is introduced as a protein, the CRISPR-based endonuclease and the guide RNA can be introduced as a protein/RNA complex. In other aspect, the guide RNA can be introduced into the cell as a DNA molecule. In such embodiments, the guide RNA coding sequence can be operably linked to promoter control sequence for expression of the guide RNA in the eukaryotic cell. For example, the RNA coding sequence can be operably linked to a promoter sequence that is recognized by RNA polymerase III (Pol III). Examples of suitable Pol III promoters include, but are not limited to, mammalian U6 or H1 promoters. Thus, in certain instances, the CRISPR-based endonuclease and the guide RNA can be introduced into the cell as DNA sequences. In some iterations, the DNA sequences encoding the CRISPR-based endonuclease and the guide RNA can be part of the same vector.

The method also comprises introducing into the cell at least one synthetic DNA sequence having a predetermined epigenetic modification. Epigenetically modified nucleic acids are detailed above in section (I). In some aspects the epigenetically modified nucleic acids can comprise additional sequences (e.g., terminal overhangs, flanking sequences with substantial sequence identity to sequences near the targeted genomic locus, flanking targeting endonuclease recognition sites, restriction endonuclease sites, insulator elements, etc.), which are detailed above in section (I).

The targeting endonuclease molecules and the epigenetically modified synthetic nucleic acid(s) can be delivered to the cell by a variety of means. In one aspect, the molecules can be delivered by a transfection method. Suitable transfection methods include nucleofection (or electroporation), calcium phosphate-mediated transfection, cationic polymer transfection (e.g., DEAE-dextran or polyethylenimine), viral transduction, virosome transfection, virion transfection, liposome transfection, cationic liposome transfection, immunoliposome transfection, nonliposomal lipid transfection, dendrimer transfection, heat shock transfection, magnetofection, lipofection, gene gun delivery, impalefection, sonoporation, optical transfection, and proprietary agent-enhanced uptake of nucleic acids. Transfection methods are well known in the art (see, e.g., “Current Protocols in Molecular Biology” Ausubel et al., John Wiley & Sons, New York, 2003 or “Molecular Cloning: A Laboratory Manual” Sambrook & Russell, Cold Spring Harbor Press, Cold Spring Harbor, N.Y., 3^{rd} edition, 2001). In another aspect, the molecules can be delivered to the cell by microinjection. For example, the molecules can be microinjected into the nucleus or cytoplasm of the cell.

The targeting endonuclease molecules and the epigenetically modified synthetic nucleic acid can be delivered to the cell simultaneously or sequentially. The ratio of the targeting endonuclease molecules to the epigenetically modified synthetic nucleic acid can range from about 1:10 to about 10:1. In various aspects, the ratio of the targeting endonuclease molecules to the epigenetically modified synthetic nucleic acid can be about 1:10, 1:9, 1:8, 1:7, 1:6, 1:5, 1:4, 1:3, 1:2, 1:1, 2:1, 3:1, 4:1, 5:1, 6:1, 7:1, 8:1, 9:1, or 10:1. A non-limiting exemplary ratio is about 1:1.

(c) Modification Using Zinc Finger Nucleases

The epigenetically modified synthetic nucleic acid(s) can be integrated into the genome of cells using zinc finger nucleases. As detailed above, the method comprises (a) introducing into the cell (i) at least one zinc finger nuclease or nucleic acid encoding the at least one zinc finger, wherein each zinc finger is engineered to recognize and introduce a double-stranded break a targeted site in the genome of the cell, and (ii) at least one synthetic epigenetically modified synthetic nucleic acid for insertion into the genome, and (b) incubating the cell such that, upon repair of the double-stranded break(s) created by the zinc finger nuclease(s), the epigenetically modified synthetic sequence is inserted into the genome of the cell.

In one aspect, the epigenetically modified synthetic nucleic acid is flanked by overhangs that are compatible with those generated by the zinc finger nuclease. For example, the epigenetically modified synthetic nucleic acid comprising the overhangs can be introduced as a linear oligonucleotide or it can be generated in situ when the epigenetically modified synthetic nucleic acid is part of a larger polynucleotide in which the epigenetically modified synthetic nucleic acid is flanked by target sites that are recognized by the zinc finger nuclease. In either case, one zinc finger nuclease can be used to introduce one double-stranded break at a targeted site in the genome, and the epigenetically modified synthetic nucleic acid can be inserted into the site by direct ligation mediated by a non-homology, end-joining DNA repair process. Insertion of the epigenetically modified synthetic nucleic acid into the genomic location disrupts or inactivates the endogenous chromosomal sequence. Alternatively, two zinc finger nucleases can be used to introduce two double-stranded breaks in the genome, and the epigenetically modified synthetic nucleic acid can be exchanged with the endogenous chromosomal sequence (which is excised and deleted).

In another aspect, the epigenetically modified synthetic nucleic acid is flanked by an upstream and a downstream sequence having substantial sequence identity with upstream and downstream sequences, respectively, of the targeted cleavage site. For example, one zinc finger nuclease can be used to introduce one double-stranded break at a targeted site in the genome, wherein, upon repair of the double-stranded break by a homology-directed DNA repair process, the epigenetically modified synthetic nucleic acid is inserted into or exchanged with a portion of the endogenous chromosomal sequence. Alternatively, a first zinc finger nuclease can be used to insert a epigenetically modified synthetic nucleic acid at a first locus by a homology-directed process as detailed immediately above, and a second zinc finger nuclease can be used to introduce a double-stranded break at a second locus, wherein the break at the second locus can be repaired by an error-prone, non-homology end-joining repair process in which an inactivating mutation is introduced at the second locus. The inactivating mutation can be a deletion of at least one nucleotide, an insertion of at least one nucleotide, a substitution of at least one nucleotide, or combinations thereof.

In any of the above-mentioned iterations, the epigenetically modified synthetic nucleic acid can replace the corresponding endogenous chromosomal sequence. Alternatively, the epigenetically modified synthetic nucleic acid can be inserted at a safe harbor locus or site that confers stability to the epigenetic modification. In this iteration, the endogenous chromosomal sequence corresponding to the epigenetically modified synthetic nucleic acid can be deleted or inactivated (as detailed herein).

(d) Modification Using CRISPR-Based Endonucleases

The epigenetically modified synthetic nucleic acid also can be inserted into the genome of a cell using CRISPR-based endonucleases. The method comprises (a) introducing into the cell (i) at least one CRISPR-based endonuclease or nucleic acid encoding the at least one CRISPR-based endonuclease, wherein each CRISPR-based endonuclease is able to cleave at least one strand of a targeted genomic sequence, (ii) at least one guide RNA or DNA encoding the at least one guide RNA, wherein the each guide RNA directs a CRISPR-based endonuclease to a targeted site in the genome, and (iii) at least one epigenetically modified synthetic nucleic acid for insertion into the genome, and (b) incubating the cell such that the epigenetically modified synthetic nucleic acid is inserted into the genome during DNA repair.

In some aspects, the CRISPR-based endonuclease contains two functional nuclease domains such that it cleaves both strands of a double-stranded sequence. For example, one CRISPR-based nuclease (or coding nucleic acid) and one guide RNA (or encoding DNA) can be introduced into the cell (along with the epigenetically modified synthetic nucleic acid). In cases in which the epigenetically modified synthetic nucleic acid is flanked by overhangs compatible with those generated by the CRISPR-based nuclease, the epigenetically modified synthetic nucleic acid can be directly ligated with the chromosomal DNA by a nonhomology-based repair process. In cases in which the epigenetically modified synthetic nucleic acid with the epigenetic modification is flanked by an upstream and a downstream sequence that share substantial sequence identity with upstream and downstream sequences, respectively, of the targeted cleavage site, the epigenetically modified synthetic nucleic acid can be inserted into or exchanged with a portion of the endogenous chromosomal sequence by a homology-directed repair process.

In other aspects, the CRISPR-based endonuclease is modified to contain one functional nuclease domain such that it cleaves one strand of a double-stranded sequence (therefore, it is a nickase). A CRISPR-based nickase can be used with two different guide RNAs to introduce nicks in the opposite strands of a double-stranded sequence, wherein the two nicks are in close enough proximity to constitute a double-stranded break. To mediate this cleavage, the two guide RNAs are oriented in a 5′-facing-5′ configuration (i.e., the upstream guide RNA binds to the sense strand of the genomic target, and the downstream guide RNA binds to the antisense strand of the genomic target). Thus, the method can comprise introducing into the cell one CRISPR-based nickase (or encoding nucleic acid), two guide RNAs (or encoding DNA), and the epigenetically modified synthetic nucleic acid. In cases in which the epigenetically modified synthetic nucleic acid is flanked by overhangs compatible with those generated by the CRISPR-based nickase system, the epigenetically modified synthetic nucleic acid can be directly ligated with the chromosomal DNA by a nonhomology-based repair process. In cases in which the epigenetically modified synthetic nucleic acid is flanked by an upstream and a downstream sequence that share substantial sequence identity with upstream and downstream sequences, respectively, of the targeted cleavage site, the epigenetically modified synthetic nucleic acid can be inserted into or exchanged with a portion of the endogenous chromosomal sequence by a homology-directed repair process.

In still other aspects, a CRISPR-based nuclease (or encoding nucleic acid) and two guide RNAs (or encoding DNA) can be introduced into the cell to mediate two double-stranded breaks in the genomic sequence. Similarly, a CRISPR-based nickase (or encoding nucleic acid) and four guide RNAs can be introduced into the cell to mediate two double-stranded breaks in the genomic sequence. In instances in which the epigenetically modified synthetic nucleic acid is flanked by overhangs that are compatible with those generated by the CRISPR-based protein, the epigenetically modified synthetic nucleic acid can be directly ligated with the chromosomal sequence, thereby replacing endogenous chromosomal sequence with epigenetically modified synthetic sequence. In iterations in which the epigenetically modified synthetic nucleic acid is flanked by an upstream and a downstream sequence having substantial sequence identity with upstream and downstream sequences, respectively, of the targeted cleavage site, the epigenetically modified synthetic nucleic acid can be inserted into or exchange with chromosomal sequence by a homology directed repair process. Alternatively, the epigenetically modified synthetic sequence can be inserted into one of the double-stranded break sites by a homology-directed repair process and the other site of double-stranded break can be mutated or inactivated by a non-homology repair process by introduction of an inactivating mutation (i.e., deletion, insertion, substitution or at least one nucleotide).

In any of the CRISPR-based endonuclease-mediated iterations, the epigenetically modified synthetic nucleic acid can replace the corresponding endogenous chromosomal sequence. Alternatively, the epigenetically modified synthetic nucleic acid can be inserted at a safe harbor locus or site that confers stability to the epigenetic modification. In this iteration, the endogenous chromosomal sequence corresponding to the epigenetically modified synthetic nucleic acid can be deleted or inactivated (as detailed herein).

### IV. Uses

The synthetic nucleic acids having predetermined epigenetic modification and cells comprising said nucleic acids have several uses. In certain aspects, engineered cells harboring insertions of epigenetically modified nucleic acids which modify the epigenetic status of regulatory regions can be used to control or alter gene expression. For example, cells having insertion of epigenetically modified nucleic acids (such as methylated nucleic acids) in addition to or in place of regulatory chromosomal sequence not normally modified (i.e., not normally methylated or hypermethylated) can be used to alter gene expression. Conversely, the replacement of endogenous regulatory sequence known to have epigenetic modifications with a synthetic nucleic acid devoid of epigenetic modifications or the insertion of a synthetic nucleic acid devoid of epigenetic modifications can be used to alter gene expression.

In another aspect, cells comprising epigenetically modified synthetic sequences in which the epigenetic modification is stable can serve as diagnostic or genotyping standards. For example, the epigenetically modified synthetic nucleic acid or cells comprising said nucleic acids can be used as reference standards in assays for diagnosing disease (such as cancer), predicting the outcome of disease, monitoring disease behavior, determining an appropriate therapy for the disease, and measuring response to targeted therapy. The provision of such reference standards in engineered cell lines is advantageous in that (1) they provide a DNA assay template within a native cellular and genomic context that undergoes all subsequent diagnostic processing steps of cell lysis (or FFPE extraction), DNA isolation, and amplification, and (2) the genetic or epigenetic alteration can be modeled into a cell type that is stable and provides large quantities of the genomic DNA.

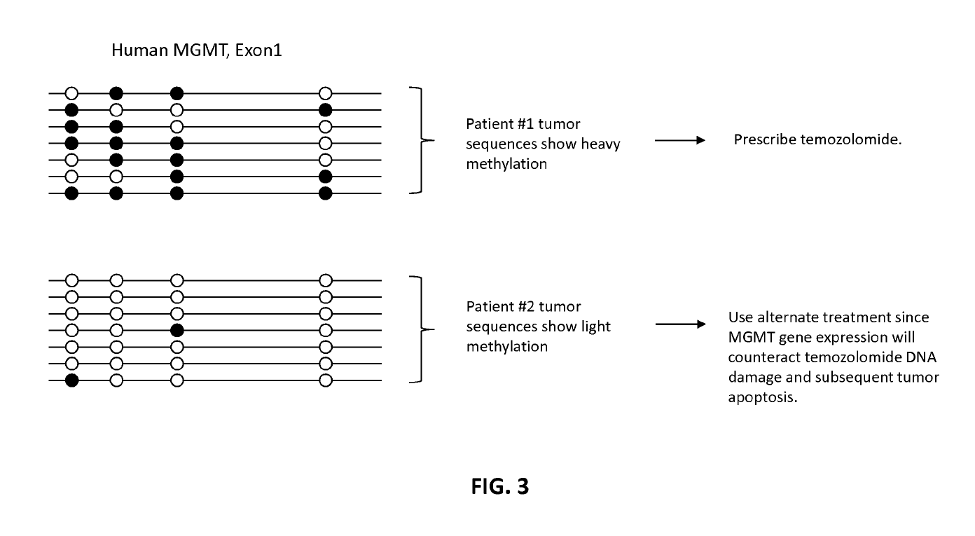

For example, MGMT expression has been shown to be useful as a prognostic and/or predictive marker in glioblastoma patients for treatment with alkylating agents. Expression of MGMT is correlated with poor outcome for treatment with alkylating agents such as temozolomide because the MGMT enzyme counters the DNA damage caused by the alkylating agent. The methylation pattern of the MGMT promoter can be used as an indicator of MGMT expression. Thus, patients having high levels of methylation at the MGMT promoter may benefit from temozolomide treatment, whereas patient with low levels of MGMT promoter methylation may not respond to temozolomide (FIG. 3). See, e.g., Hegi et al., 2004, Clin. Cancer Res. 10(6):1871-4; Hegi et al., 2005, New England J. Med. 352(10): 997-1003; Boots-Sprenger et al., 2013, Modern Pathol. 26(7): 922-9. In yet another case, methylation at the BRCA1 gene has been used in combination with loss of heterozygosity (LOH) measurements to predict if certain ovarian cancers are candidates for treatment with PARP inhibitors or platinum salts (Abkevich et al., 2012, Br J Cancer 107(10):1776-82. Thus, engineered cells comprising hyper- or hypo-methylated MGMT or BRCA1 sequences can be used as reference standards for assessing methylation status. Additionally, in the absence of patient samples having well-characterized levels methylation, engineered cells with targeted methylation patterns can serve as control samples to develop and characterize new detection assays or as quality control measures in the set-up or maintenance of research or diagnostic labs.

In a further aspect, engineered cells comprising epigenetically modified sequences in which the epigenetic modification is metastable can be used to analyze the epigenetic stability of a modified sequence in a cell based on a priori knowledge of the epigenetic modification pattern or status of the inserted sequence. For example, said sequences can be used to analyze the epigenetic stability of the locus in response to drug, environmental, or dietary factors. In particular, an artificially methylated locus can serve as a starting point to “reset” the methylation pattern and study what biological factors result in subsequent methylation and gene expression changes. As an example, there is a well-known association between weight and coat color changes and the methylation status of the murine Agouti gene following dietary supplementation (Dolinoy et al., 2007, Pediatric Research, 61: 30R). Since chemically modified sequences can be inserted at precise locations using targeting endonucleases (as detailed above), said modified sequences can be placed into a native chromosomal environment that remains subject to any locus specific epigenetic regulation factors that may be unique to the chromosomal region of interest. Such locus-specific experimentation using ectopic methylated DNA sequences is not possible.

In still another aspect, engineered cells comprising epigenetically modified synthetic sequences can be used as a source of genomic DNA comprising the epigenetically modified sequence. For example, DNA can be extracted from live or fixed cells, amplified, and analyzed using standard techniques. Alternatively, synthetic chromosomal sequences with epigenetic modification can be analyzed in situ in the cells, e.g., via in situ PCR, in situ Western, immunohistochemistry, and other suitable procedures.

### V. Kits

The invention further provides kits comprising the epigenetically modified synthetic sequences and/or cells comprising said sequences described herein. In one embodiment, a kit is provided for predicting responsiveness of a disease in a subject to a therapeutic treatment or regimen, such as a cancer therapy, which kit includes at least one synthetic nucleic acid having a predetermined cytosine modification that correlates with known treatment outcome along with documents for interpretation of comparison of the reference standard (i.e., the epigenetically modified synthetic sequence) with a sample taken from the subject. The kit may further comprise a control chromosomal sequence. In another embodiment, a kit is provided for diagnosing disease in a subject sample, which kit includes at least one synthetic nucleic acid having a predetermined cytosine modification that correlates with known disease state along with documents for interpretation of comparison of the reference standard with a sample taken from the subject. The kit may further comprise a control chromosomal sequence. In another embodiment, a kit is provided for predicting outcome or severity of a disease in a subject sample, which kit includes at least one synthetic nucleic acid having a predetermined cytosine modification that correlates with known prognosis of the disease along with documents for interpretation of comparison of the reference standard with a sample taken from the subject. The kit may further comprise a control chromosomal sequence.

In other embodiments, the kit includes a panel of multiple epigenetically modified synthetic sequences, wherein each synthetic sequence of the kit has a different predetermined cytosine modification correlated with a different known (1) level of sensitivity to a disease treatment, (2) diagnosis of a disease, or (3) prognosis of a disease. Such panel provides multiple standards against which cytosine modification(s) of a subject's sample can be compared to (1) determine diagnosis of disease, or (2) determine prognosis of disease, or (3) assess the outcome of a treatment regimen. In any of these different kits, the kit may further comprise one or more control chromosomal sequence as well as documents for interpretation of comparison of the reference standards with a sample taken from the subject.

In some aspects, the epigenetically modified synthetic sequence or sequences are provided in one or more fixed cells. When synthetic sequences having multiple cytosine modifications are provided, whether or not they are incorporated in cells, the samples will be provided in separate, clearly labeled packaging.

### DEFINITIONS

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

As used interchangeably herein, the term “CpG location” and “CpG site” refer to regions of DNA where a cytosine nucleotide occurs next to a guanine nucleotide in the linear sequence of bases along its length, where “CpG” is an abbreviation for a “—C-phosphate-G-” linkage, i.e. cytosine and guanine separated by a single phosphate. The term “CpG island” refers to a cluster of CpG sites.

As used herein, the term “endogenous sequence” refers to a chromosomal sequence that is native to the cell.

The term “exogenous sequence,” as used herein, refers to a sequence that is not native to the cell, or a chromosomal sequence whose native location in the genome of the cell is in a different chromosomal location.

A “gene,” as used herein, refers to a DNA region (including exons and introns) encoding a gene product, as well as all DNA regions which regulate the production of the gene product, whether or not such regulatory sequences are adjacent to coding and/or transcribed sequences. Accordingly, a gene includes, but is not necessarily limited to, promoter sequences, terminators, translational regulatory sequences such as ribosome binding sites and internal ribosome entry sites, enhancers, silencers, insulators, boundary elements, replication origins, matrix attachment sites, and locus control regions.

The term “heterologous” refers to an entity that is not endogenous or native to the cell of interest. For example, a heterologous protein refers to a protein that is derived from or was originally derived from an exogenous source, such as an exogenously introduced nucleic acid sequence. In some instances, the heterologous protein is not normally produced by the cell of interest.

The terms “nucleic acid” and “polynucleotide” refer to a deoxyribonucleotide or ribonucleotide polymer, in linear or circular conformation, and in either single- or double-stranded form. For the purposes of the present disclosure, these terms are not to be construed as limiting with respect to the length of a polymer. The terms can encompass known analogs of natural nucleotides, as well as nucleotides that are modified in the base, sugar and/or phosphate moieties (e.g., phosphorothioate backbones). In general, an analog of a particular nucleotide has the same base-pairing specificity; i.e., an analog of A will base-pair with T.

The term “nucleotide” refers to deoxyribonucleotides or ribonucleotides. The nucleotides may be standard nucleotides (i.e., adenosine, guanosine, cytidine, thymidine, and uridine) or nucleotide analogs. A nucleotide analog refers to a nucleotide having a modified purine or pyrimidine base or a modified ribose moiety. A nucleotide analog may be a naturally occurring nucleotide (e.g., inosine) or a non-naturally occurring nucleotide. Non-limiting examples of modifications on the sugar or base moieties of a nucleotide include the addition (or removal) of acetyl groups, amino groups, carboxyl groups, carboxymethyl groups, hydroxyl groups, methyl groups, phosphoryl groups, and thiol groups, as well as the substitution of the carbon and nitrogen atoms of the bases with other atoms (e.g., 7-deaza purines). Nucleotide analogs also include dideoxy nucleotides, 2′-O-methyl nucleotides, locked nucleic acids (LNA), peptide nucleic acids (PNA), and morpholinos.

The terms “polypeptide” and “protein” are used interchangeably to refer to a polymer of amino acid residues.

Techniques for determining nucleic acid and amino acid sequence identity are known in the art. Typically, such techniques include determining the nucleotide sequence of the mRNA for a gene and/or determining the amino acid sequence encoded thereby, and comparing these sequences to a second nucleotide or amino acid sequence. Genomic sequences can also be determined and compared in this fashion. In general, identity refers to an exact nucleotide-to-nucleotide or amino acid-to-amino acid correspondence of two polynucleotides or polypeptide sequences, respectively. Two or more sequences (polynucleotide or amino acid) may be compared by determining their percent identity. The percent identity of two sequences, whether nucleic acid or amino acid sequences, is the number of exact matches between two aligned sequences divided by the length of the shorter sequences and multiplied by 100. An approximate alignment for nucleic acid sequences is provided by the local homology algorithm of Smith and Waterman, Advances in Applied Mathematics 2:482-489 (1981). This algorithm may be applied to amino acid sequences by using the scoring matrix developed by Dayhoff, Atlas of Protein Sequences and Structure, M. O. Dayhoff ed., 5 suppl. 3:353-358, National Biomedical Research Foundation, Washington, D.C., USA, and normalized by Gribskov, Nucl. Acids Res. 14(6):6745-6763 (1986). An exemplary implementation of this algorithm to determine percent identity of a sequence is provided by the Genetics Computer Group (Madison, Wis.) in the “BestFit” utility application. Other suitable programs for calculating the percent identity or similarity between sequences are generally known in the art, for example, another alignment program is BLAST, used with default parameters. For example, BLASTN and BLASTP may be used using the following default parameters: genetic code=standard; filter=none; strand=both; cutoff=60; expect=10; Matrix=BLOSUM62; Descriptions=50 sequences; sort by=HIGH SCORE; Databases=non-redundant, GenBank+EMBL+DDBJ+PDB+GenBank CDS translations+Swiss protein+Spupdate+PIR. Details of these programs may be found on the GenBank website.

As various changes could be made in the above-described animals, cells and methods without departing from the scope of the invention, it is intended that all matter contained in the above description and in the examples given below, shall be interpreted as illustrative and not in a limiting sense.

### EXAMPLES

The following examples illustrate certain aspects of the invention.

### Example 1

### Stable Integration of Synthetically Methylated DNA

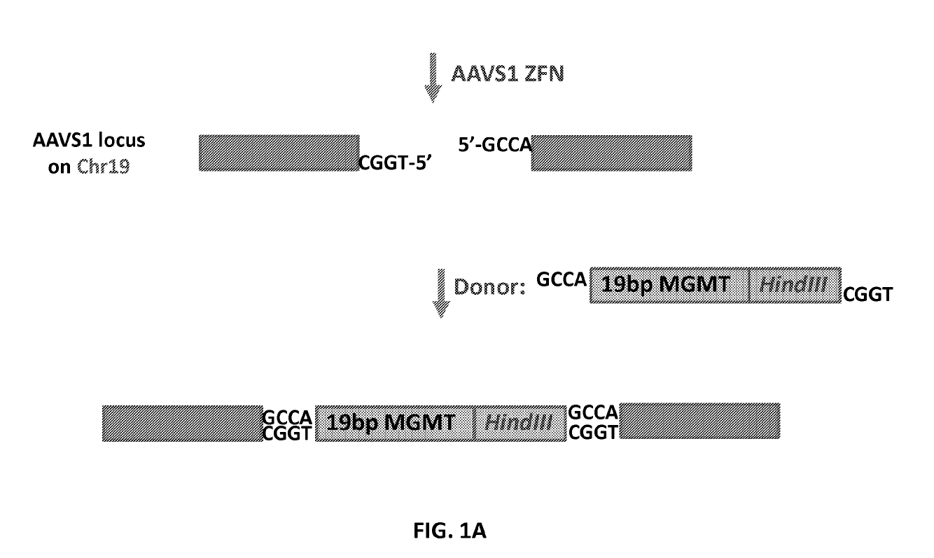

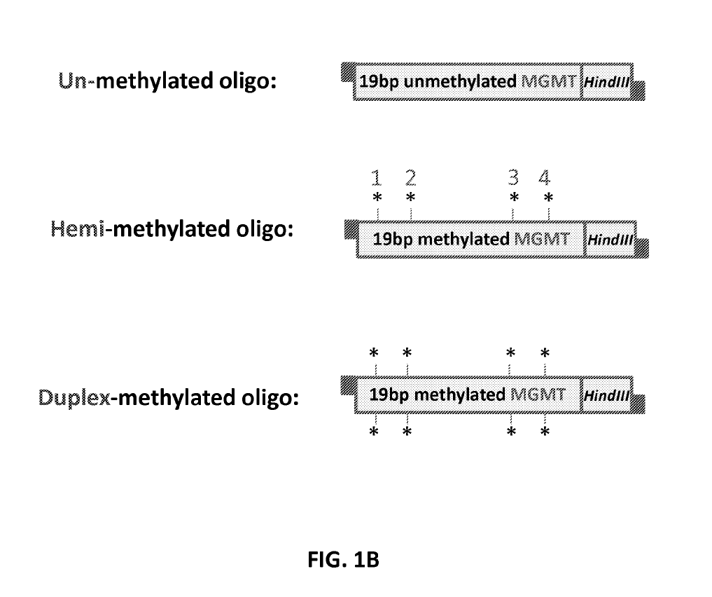

The purpose of this study was to determine whether synthetically methylated DNA can be stably integrated into the chromosomes of a cell using ZFN targeted genome modification. A fragment of human O⁶-methylguanine-DNA methyltransferase (MGMT) gene having different methylation patterns was inserted into a targeted site of the AAVS1 locus on chromosome 19 of human cells. FIG. 1A diagrams the strategy.

Two single-stranded oligodeoxynucleotides (ssODNs) comprising a 19 nt sequence from the human MGMT gene (i.e., 5′-CGACGCCCGCAGGRCCTGC-3′, SEQ ID NO:9) and a HindIII restriction endonuclease site (5′-AAGCTT-3′) for colony screening were synthesized. The CpG sites, designated 1 to 4 (from 5′ to 3′), are underlined in SEQ ID NO:9. One ssODN contained 5-methylcytosine in the CpG sites. Two complementary (methylated and unmethylated) ssODNs were also synthesized. Various combinations of the ssODNs were annealed at a final concentration of 95 μM in annealing buffer containing 5 mM Tris.HCl, pH 8.0, 0.5 mM EDTA, pH 8.0, 50 mM NaCl to form non-methylated, hemi-methylated, and duplex-methylated double-stranded oligodeoxynucleotides (dsODNs) (see FIG. 1B). The overhangs on the dsODNs were designed to be compatible with the 5′-GCCA-3′ overhangs created by the FokI enzyme at the site of cleavage of a zinc finger nuclease targeting the human AAVS1 locus.

One million human K562 cells in a volume of 100 μl were nucleofected with 3.2 μL of 95 μM dsODNs along with 5.0 μg of AAVS1 ZFN RNAs in triplicate experiments. Aliquots of cells were harvested on days 2, 6, 8, and 10 following nucleofection, PCR was used to amplify the region that harbors the integrated sequence, and the PCR products were subjected to HindIII digestion. Fragments of 264 and 180 bp were detected under each condition (i.e., non-methylated dsODN+ZFN, hemi-methylated dsODN+ZFN, and duplex-methylated dsODN+ZFN) on each day.

After 20 days in culture, the nucleofected cells were FAC sorted for single living cells and seeded on 96-well plates. Thirty-five days after nucleofection, cells derived from each single cell colony were partitioned in two portions: one portion was frozen and the other portion was to screen for integration of the dsODN sequence. Genomic DNA was extracted, the AAVS1 region harboring the integrated sequence was PCR amplified, and the PCR products were subjected to RFLP analysis by HindIII enzyme digestion. Approximately 1300 colonies were screened by HindIII digestion, and those with HindIII fragments of the proper size were subjected to regular DNA sequencing. A total of 18 single-cell clones were identified with correct insertion of the 25 bp fragment (i.e., 19 bp MGMT fragment and HindIII site) into one of the three alleles of the AAVS1 locus. Specifically, 5 colonies has correct integration of the un-methylated insertion; 4 colonies has correct integration of the hemi-methylated insertion; and 9 colonies had has correct integration of the duplex-methylated insertion. The sequence of the modified AAVS1 locus was 5′-CCTTACCTCTCTAGTCTGTGCTAGCTCTTCCAGCCCCCTGTCATGGCATCTTCCAGG GGTCCGAGAGCTCAGCTAGTCTTCTTCCTCCAACCCGGGCCCCTATGTCCACTTCA GGACAGCATGTTTGCTGCCTCCAGGGATCCTGTGTCCCCGAGCTGGGACCACCTT ATATTCCCAGGGCCGGTTAATGTGGCTCTGGTTCTGGGTACTTTTATCTGTCCCCTC CACCCCACAGTGGGGCCACGACGCCCGCAGGTCCTCGAAGCTTGCCACTAGGGA CAGGATTGGTGACAGAAAAGCCCCATCCTTAGGCCTCCTCCTTCCTAGTCTCCTGA TATTGGGTCTAACCCCCACCTCCTGTTAGGCAGATTCCTTATCTGGTGACACACCCC CATTTCCTGGAGCCATCTCTCTCCTTGCCAGAACCTCTAAGGTTTGCTTACG-3′ (SEQ ID NO:10; 25 bp insert shown in bold, HindIII site shown in italics). These data show that synthetically methylated DNA can be stably integrated into the genome of human cells.

### Example 2

### Stable Maintenance of Synthetically Methylated DNA

The purpose of this study was to determine whether the methylation status of synthetically methylated DNA integrated into a genome can be stably maintained. Nine cell colonies with correct insertion of the MGMT fragment in each of the three alleles of the AAVS1 locus (see Example 1) were regrown for two weeks. The methylation status of each colony was determined by pryosequencing (EpigenDx, Hopkinton, Mass.). The methylation analysis is at 49 day post nucelofection is shown in Table. 1.

TABLE 1
Methylation Analysis at 49 days after nucleofection.

| ID | Methylation | Methylation percentage (%) | | | | Overall Region | |
|---|---|---|---|---|---|---|---|
| # | Status | CpG#1 | CpG#2 | CpG#3 | CpG#4 | Mean | SD |
| 1 | Non- | 0.0 | 0.0 | 3.9 | 0.0 | 1.0 | 1.9 |
| 2 | Non- | 2.7 | 4.6 | 7.8 | 3.8 | 4.7 | 2.2 |
| 3 | Non- | 0.0 | 3.8 | 8.5 | 3.3 | 3.9 | 3.5 |
| 4 | Hemi- | 0.0 | 2.9 | 8.0 | 2.5 | 3.4 | 3.4 |
| 5 | Hemi- | 0.0 | 0.0 | 7.1 | 2.8 | 2.5 | 3.4 |
| 6 | Hemi- | 3.2 | 6.6 | 17.7 | 4.7 | 8.0 | 6.6 |
| 7 | Duplex- | 15.3 | 28.4 | 35.8 | 21.8 | 25.3 | 8.8 |
| 8 | Duplex- | 0.0 | 2.6 | 9.2 | 5.1 | 4.2 | 3.9 |
| 9 | Duplex- | 4.0 | 3.1 | 11.4 | 3.3 | 5.5 | 4.0 |

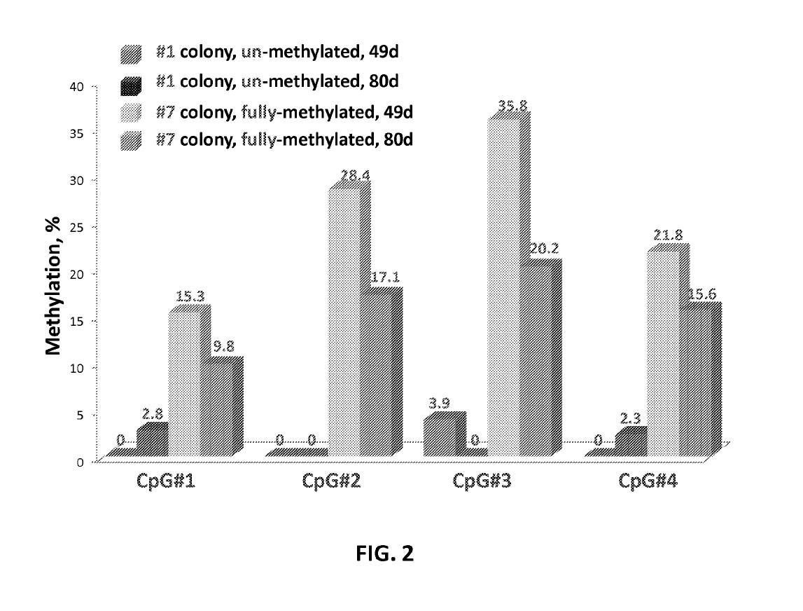

Duplicates (A, B) of colony #1 (non-methylated) and colony #7 (duplex-methylated) were grown for an additional 31 days. The methylation status (at 80 days post nucelofection) of each colony was determined by pryosequencing, and is shown in Table. 2. FIG. 2 summarizes the methylation status at each CpG sites in these two different alleles at days 49 and 80 post transfection. These data show that the methylation status can be transmitted from the synthetic DNA to the genomic locus, and that predetermined patterns of DNA methylation largely can be maintained from generation to generation.

TABLE 2
Methylation Analysis at 80 days after nucleofection.

| ID | Methylation | Methylation percentage (%) | | | | Overall Region | |
|---|---|---|---|---|---|---|---|
| # | Status | CpG#1 | CpG#2 | CpG#3 | CpG#4 | Mean | SD |
| 1A | Non- | 2.3 | 0.0 | 0.0 | 2.0 | 1.1 | 1.2 |
| 1B | Non- | 3.3 | 0.0 | 0.0 | 2.6 | 1.5 | 1.7 |
| 7A | Duplex- | 10.0 | 17.8 | 19.1 | 16.8 | 15.9 | 4.1 |
| 7B | Duplex- | 9.5 | 16.4 | 21.2 | 18.3 | 16.4 | 5.0 |

### Example 3

### Uses of Cells Having Stable MGMT Methylation Patterns

Cells having stable MGMT methylation patterns (such as those prepared above) can be used as diagnostic controls in assays for determining an appropriate course of treatment for patients suffering from glioblastoma. The level of MGMT promoter methylation in patient tumor samples can be analyzed and compared to that of the control (reference) cells with the stable MGMT. For example, DNA can be extracted from tumor and control samples using standard procedures. The extracted DNA can be treated with bisulfite, amplified using methylation-specific PCR, and sequenced. Alternatively, the methylation status of the extracted DNA can be determined using pyrosequencing. Alternatively, the methylation status of the MGMT promoter can be analyzed by immunohistochemistry in fixed cells using a methylation specific antibody raised against MGMT. The methylation status of patient samples then can be compared to that of the control cells. If the methylation level of the sample taken from the patient is lower than that of the control cells, then the tumor is deemed to be negative for MGMT methylation, and temozolomide is not administered. If the methylation level of the sample taken from the patient is equal to or greater than that of the control cells, then the tumor is deemed to be positive for MGMT methylation, and temozolomide is administered (see FIG. 3).

1. A genetically modified cell line comprising at least one chromosomally integrated nucleic acid having a predetermined cytosine modification, wherein the cytosine modification is correlated with a known diagnosis, prognosis, or level of sensitivity to a disease treatment.

2. The genetically modified cell line of claim 1, wherein the cytosine modification is chosen from 5-methylcytosine (5mC), 3-methylcytosine (3mC), 5-hydroxymethylcytosine (5hmC), 5-formylcytosine (5fC), or 5-carboxylcytosine (5caC).

3. The genetically modified cell line of claim 1, wherein the chromosomally integrated nucleic acid has a sequence with substantial sequence identity to that of a control element, a portion of a control element, a coding region, or a portion of a coding region of a gene associated with a disease.

4. The genetically modified cell line of claim 3, wherein the gene is a gene listed in Table 1.

5. The genetically modified cell line of claim 4, wherein the gene is chosen from MGMT, BRCA1, BRCA2, PITX2, GSTP1, APC, RASSF1, or HER2.

6. The genetically modified cell line of claim 1, wherein the chromosomally integrated nucleic acid is inserted into a chromosomal location in the cell using a targeting endonuclease.

7. The genetically modified cell line of claim 6, wherein the targeting endonuclease is chosen from a zinc finger nuclease, a CRISPR-based endonuclease, a meganuclease, a transcription activator-like effector nuclease (TALEN), a I-TevI nuclease or related monomeric hybrid, or an artificial targeted DNA double strand break inducing agent.

8. The genetically modified cell line of claim 1, wherein the chromosomally integrated nucleic acid replaces an endogenous chromosomal sequence from which the chromosomally integrated nucleic acid is derived.

9. The genetically modified cell line of claim 1, wherein the chromosomally integrated nucleic acid is inserted at a locus possessing adjacent insulating elements or other elements that assist in maintaining the original cytosine modification status of the chromosomally integrated nucleic acid.

10. The genetically modified cell line of claim 9, wherein the locus is chosen from AAVS1, CCR5, HPRT, or ROSA26.

11. The genetically modified cell line of claim 9, wherein endogenous chromosomal sequence corresponding to the chromosomally integrated nucleic acid is inactivated or deleted.

12. The genetically modified cell line of claim 1, further comprising at least one nucleic acid sequence encoding a recombinant protein.

13. The genetically modified cell line of claim 1, wherein the cell line is a human cell line.

14. The genetically modified cell line of claim 1, wherein the predetermined cytosine modification is stable.

15. The genetically modified cell line of claim 1, wherein the predetermined cytosine modification is metastable.

16. A kit for diagnosing a disease or predicting prognosis for a disease in a subject or for predicting responsiveness of a disease in a subject to a therapeutic treatment, the kit comprising at least one nucleic acid having a predetermined cytosine modification for use as a reference standard, wherein the cytosine modification is correlated with a known diagnosis, prognosis, or level of sensitivity to a disease treatment.

17-18. (canceled)

19. The kit of claim 16 comprising at least two nucleic acids, wherein each nucleic acid has a different predetermined cytosine modification.

20. The kit of claim 16, wherein the cytosine modification is chosen from 5-methylcytosine (5mC), 3-methylcytosine (3mC), 5-hydroxymethylcytosine (5hmC), 5-formylcytosine (5fC), or 5-carboxylcytosine (5caC).

21. The kit of claim 16, wherein the nucleic acid has a sequence with substantial sequence identity to that of a control element, a portion of a control element, a coding region, or a portion of a coding region of a gene associated with a disease.

22. The kit of claim 21, wherein the gene is a gene listed in Table 1.

23. The kit of claim 22, wherein the gene is chosen from MGMT, BRCA1, BRCA2, PITX2, GSTP1, APC, RASSF1, or HER2.

24. The kit of claim 16, wherein the nucleic acid is located within a chromosome of a cell.

25. The kit of claim 24, wherein the cell is a fixed cell.

26. The kit of claim 24, wherein the nucleic acid is within genomic DNA isolated from the cell.


SEQUENCE LISTING (10 sequences)

SEQ ID NO: 1 - protein, 7 aa, Artificial Sequence
  SYNTHESIZED
PKKKRKV

SEQ ID NO: 2 - protein, 7 aa, Artificial Sequence
  SYNTHESIZED
PKKKRRV

SEQ ID NO: 3 - protein, 16 aa, Artificial Sequence
  SYNTHESIZED
KRPAATKKAGQAKKKK

SEQ ID NO: 4 - protein, 20 aa, Artificial Sequence
  SYNTHESIZED
GRKKRRQRRRPPQPKKKRKV

SEQ ID NO: 5 - protein, 19 aa, Artificial Sequence
  SYNTHESIZED
PLSSIFSRIGDPPKKKRKV

SEQ ID NO: 6 - protein, 24 aa, Artificial Sequence
  SYNTHESIZED
GALFLGWLGAAGSTMGAPKKKRKV

SEQ ID NO: 7 - protein, 27 aa, Artificial Sequence
  SYNTHESIZED
GALFLGFLGAAGSTMGAWSQPKKKRKV

SEQ ID NO: 8 - protein, 21 aa, Artificial Sequence
  SYNTHESIZED
KETWWETWWTEWSQPKKKRKV

SEQ ID NO: 9 - DNA, 19 bp, Homo sapiens
cgacgcccgcaggtcctcg

SEQ ID NO: 10 - DNA, 444 bp, Homo sapiens
ccttacctctctagtctgtgctagctcttccagccccctgtcatggcatcttccaggggtccgagagctcagctagtcttcttcctccaacccgggcccctatgtccacttcaggacagcatgtttgctgcctccagggatcctgtgtccccgagctgggaccaccttatattcccagggccggttaatgtggctctggttctgggtacttttatctgtcccctccaccccacagtggggccacgacgcccgcaggtcctcgaagcttgccactagggacaggattggtgacagaaaagccccatccttaggcctcctccttcctagtctcctgatattgggtctaacccccacctcctgttaggcagattccttatctggtgacacacccccatttcctggagccatctctctccttgccagaacctctaaggtttgcttacg